(19) United States
(12) Patent Application Publication
(10) Pub. No.: US 2025/0107896 A1
(43) Pub. Date: Apr. 3, 2025

(54) Anchor for an Implantable Joint Replacement Device

(71) Applicant: Djit Medtech, Inc., Baltimore, MD (US)

(72) Inventors: Marcia Andrews Hart, Baltimore, MD (US); Ryan David Katz, Baltimore, MD (US); Fraser M. Smith, Salt Lake City, UT (US)

(21) Appl. No.: 18/825,887

(22) Filed: Sep. 5, 2024

Related U.S. Application Data
(60) Provisional application No. 63/587,136, filed on Oct. 1, 2023.

(51) Int. Cl. A61F 2/30; A61F 2/42

(52) U.S. Cl. CPC A61F 2/30749; A61F 2/4225; A61F 2/4241; A61F 2002/30116; A61F 2002/30235; A61F 2002/30392; A61F 2002/30593; A61F 2/30756; A61F 2002/30841; A61F 2002/30858; A61F 2002/30861; A61F 2002/30878; A61F 2002/4228; A61F 2002/4243; A61F 2220/0016

(57) ABSTRACT

An anchor for an implantable device for arthroplasty can include a hollow anchor body defining an interior cannula space. A slot can extend along a length of the anchor body. The slot can provide an opening from the interior cannula space to an exterior of the anchor body. The slot can have a slot width that is smaller than an internal width dimension in the interior cannula space. The anchor can also include a rounded edge at a transition between the slot and at least one of an interior surface of the anchor body or an exterior surface of the anchor body.

### RELATED APPLICATIONS

This application claims the benefit of U.S. Provisional Application Ser. No. 63/587,136, filed Oct. 1, 2023, and entitled, “Anchor for an Implantable Joint Replacement Device”, which is incorporated by reference in its entirety herein.

### BACKGROUND

Joint replacement is often used to treat arthritis, including osteoarthritis, rheumatoid arthritis, and arthritis due to traumatic injury. For example, in osteoarthritis, the degeneration of the joint can lead to bone-on-bone contact, which is a common cause of severe pain in the advanced stages of all forms of arthritis. Bone-on-bone contact can lead to inefficient joint mechanics that impair range of motion, accelerate the degenerative process, and may ultimately lead to an ankylosis or complete loss of motion at the joint. Interphalangeal arthroplasty, or replacement of joints between the small bones of the fingers or toes, may be used to treat arthritis in the hands and feet.

Fusion of the joint can be an alternative to arthroplasty, but this may be undesirable because of the loss of motion of the joint. Fusion involves affixing two bones in a specific position so that the bones fuse together into a single osseous unit that can be stable and pain-free. Despite the fact that the motion of the joint is lost, fusion is a popular treatment for joints of the fingers and toes because it can be difficult to provide durable and reliable arthroplasty alternatives.

Some implants for arthroplasty that have been used include stems that extend longitudinally into the phalanges of the finger or toe. These devices are often implanted through a longitudinal incision on the dorsal aspect of the finger or toe. Other implants can include two unconstrained parts that replace surfaces of the phalanges on either side of a joint. The two parts are not connected in a way that constrains the joint, so this type of implant relies on the strength of ligaments and tendons to hold the surfaces in place. Another type of implant can replace the surface of one phalange while leaving the neighboring phalange unmodified. This can be used when only one of the bones has been damaged.

### BRIEF DESCRIPTION OF THE DRAWINGS

Features and advantages of the invention will be apparent from the detailed description which follows, taken in conjunction with the accompanying drawings, which together illustrate, by way of example, features of the invention; and, wherein:

FIG. 1A is a side view of an example flexible connector for an implantable device for arthroplasty.

FIG. 1B is an isometric view of the example flexible connector of FIG. 1 for an implantable device for arthroplasty.

FIG. 1C is another side view of the example flexible connector of FIG. 1 for an implantable device for arthroplasty.

FIG. 1D is another perspective view of the example flexible connector of FIG. 1 for an implantable device for arthroplasty.

FIG. 2 is an isometric view of another example flexible connector for an implantable device for arthroplasty.

FIG. 3 is an isometric view of another example flexible connector for an implantable device for arthroplasty.

FIG. 4 is a side view of another example flexible connector for an implantable device for arthroplasty.

FIGS. 5A-5L are end-on views of anchoring portions of example flexible connectors that have various non-uniform thickness profiles.

FIG. 6 is an exploded view of an example implantable device for arthroplasty.

FIG. 7 is a partial cutaway view of an example flexible connector for an implantable device for arthroplasty.

FIG. 8 is a partial cutaway view of another example flexible connector for an implantable device for arthroplasty.

FIGS. 9A and 9B are partial cutaway views of an example flexible connector for an implantable device for arthroplasty.

FIGS. 10A and 10B are partial cutaway views of an example flexible connector for an implantable device for arthroplasty.

FIG. 11 is another partial cutaway view of an example flexible connector for an implantable device for arthroplasty.

FIG. 12 is another partial cutaway view of an example flexible connector for an implantable device for arthroplasty.

FIG. 13 is another partial cutaway view of an example flexible connector for an implantable device for arthroplasty.

FIG. 14 is another partial cutaway view of an example flexible connector for an implantable device for arthroplasty.

FIG. 15 is another partial cutaway view of an example flexible connector for an implantable device for arthroplasty.

FIG. 16 is another partial cutaway view of an example flexible connector for an implantable device for arthroplasty.

FIG. 17 is an isometric view of an example fixation rod operable with a flexible connector for an implantable device for arthroplasty.

FIG. 18 is an isometric view of another example fixation rod operable with a flexible connector for an implantable device for arthroplasty.

FIG. 19 is a partial cutaway view of an example implantable device for arthroplasty.

FIG. 20A is a top-down cutaway view of an example implantable device for arthroplasty implanted in a joint.

FIG. 20B is an isometric view of the flexible connector used in FIG. 20A.

FIG. 21 is a side view of an example flexible connector for an implantable device for arthroplasty.

FIG. 22 is a side view of another example flexible connector for an implantable device for arthroplasty.

FIG. 23A is a side cross-sectional view of an example flexible connector for an implantable device for arthroplasty.

FIG. 23B is a cutaway view of the example flexible connector for an implantable device for arthroplasty of FIG. 23A.

FIG. 24 is a side view of another example flexible connector for an implantable device for arthroplasty.

FIG. 25 is a side view of another example flexible connector for an implantable device for arthroplasty.

FIG. 26A is a side view of an example flexible connector between two phalanges.

FIG. 26B is an isometric view of the flexible connector of FIG. 26A.

FIG. 27A is a side view of an example flexible connector for an implantable device for arthroplasty.

FIG. 27B is an exploded view of the example flexible connector for an implantable device for arthroplasty of FIG. 27A.

FIGS. 28A-28D are side views of example flexible connectors for an implantable device for arthroplasty.

FIG. 29A is a side view of an example anchor for an implantable device for arthroplasty.

FIG. 29B is an isometric view of the example anchor for an implantable device for arthroplasty of FIG. 29A.

FIGS. 30A-30L are cross-sectional views of example anchors having interior cannula spaces with various non-uniform diameter profiles.

FIG. 31A is an isometric view of an example anchor for an implantable device for arthroplasty.

FIG. 31B is a side view of another example anchor for an implantable device for arthroplasty.

FIG. 32A is an isometric view of another example anchor for an implantable device for arthroplasty.

FIG. 32B is a side view of another example anchor for an implantable device for arthroplasty.

FIG. 33 is an isometric view of another example anchor for an implantable device for arthroplasty.

FIG. 34 is an isometric view of another example anchor for an implantable device for arthroplasty.

FIG. 35 is a cutaway view of an example implantable device for arthroplasty implanted in a joint.

FIGS. 36A-36D show aspects of another example implantable device for arthroplasty implanted in a joint.

FIG. 37 is an isometric view of phalanges with example anchors implanted therein.

FIG. 38 is an isometric view of phalanges with example anchors implanted therein and a bone saw for forming slots in the phalanges.

FIG. 39 is an isometric view of a replacement of a flexible connector with a replacement flexible connector into two anchors.

Reference will now be made to the exemplary embodiments illustrated, and specific language will be used herein to describe the same. It will nevertheless be understood that no limitation of the scope of the invention is thereby intended.

### DETAILED DESCRIPTION

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness can in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

As used herein, “adjacent” refers to the proximity of two structures or elements. Particularly, elements that are identified as being “adjacent” can be either abutting or connected. Such elements can also be near or close to each other without necessarily contacting each other. The exact degree of proximity can in some cases depend on the specific context.

As used herein, “distal” and “proximal” can be understood according to the context in which they are used. In some cases, “distal to” can mean “farther away from” and a component or part of a component or a point that is described as “distal” can be farther relative to some other component or part of a component or a point relative to a point of reference. In other cases, the term “distal” can be used to indicate that an element is related to or a part of another element that is also described as “distal.” For example, a bore drilled in a “distal phalange” can be referred to as a “distal bore,” and an anchor implanted in the distal phalange can be referred to as a “distal anchor.” Similarly, proximal can mean “closer to” in some contexts. In other contexts, an element can be described as “proximal” because it is part of or related to another element that is also described as “proximal.” For example, an anchor implanted into a “proximal phalange” can be referred to as a “proximal anchor.”

As used herein, the phrase “at least one of” followed by a list of alternatives is to be interpreted as encompassing any single one of the alternatives or any combination of multiple of the alternatives. For example, “at least one of A, B, or C” includes A alone, B alone, C alone, A and B in combination, A and C in combination, B and C in combination, and a combination of A, B, and C.

An initial overview of technology embodiments is provided below and then specific technology embodiments are described in further detail later. This initial summary is intended to aid readers in understanding the technology more quickly but is not intended to identify key features or essential features of the technology nor is it intended to limit the scope of the claimed subject matter.

As explained above, several types of implants have been used for arthroplasty of interphalangeal joints. However, these implants may not be sufficient stable or reliable. The surgical procedures used for implanting the implants can also have negative effects on the joint. For example, some implants include stems that extend partially along the length of the bones. These are often implanted through an incision in the dorsal aspect of the digit. Preparing the joint through the dorsal aspect in this way can involve surgical disruption of the extensor mechanism to allow for bone preparation. Preparation of the bone includes removal of a portion of the condyle head and serially broaching the medullary canals of the phalanges to provide room for the implant stem. Thus, the patient can be negatively affected by the disruption of the extensor and removal of a significant amount of bone from the phalanges. After surgery, flexing the digit can put stress on the dorsal incision. Therefore, the digit may be immobilized for an extended period after surgery to allow the incision to heal.

Constrained stemmed silicone implants often do not match physiologic motion of a normal joint. Thus, these implants often do not provide predictable motion outcomes. Stemmed implants are also subject to loosening, implant dislocation, implant breakage such as silicone fragmentation, osteolysis and erosion through bone, and collagen encapsulation of the implant that may further restrict range of motion.

On the other hand, unconstrained implants that have two separate components can rely on the tendons and ligaments of the patient to hold the implant in place. These implants can be unstable and subject to dislocation.

Squeaking and other sounds may be produced by direct contact of the components. These implants can also be subject to loosening, osteolysis and erosion through the bone. Because of the high failure rate of joint replacement devices, fusion is often used to provide pain relief and digit stability despite the significant loss of motion caused by joint fusion.

The present disclosure describes implantable devices for arthroplasty that can address several of the issues with other joint replacement devices. In some examples, an implantable device for arthroplasty can include a flexible connector. The flexible connector can include a flexible central portion, a first anchoring portion extending from a first end of the central portion, and a second anchoring portion extending from a second end of the central portion. The first anchoring portion and the second anchoring portion can each include a thickened portion located farther away from the central portion and a thinner portion, relative to the thickened portion, located nearer to the central portion than the thickened portion. In other words, the anchoring portions can include a thickened portion farther away from the flexible central portion and a thinner portion closer to the flexible central portion. The first anchoring portion can be configured to be implanted in a cavity formed in a phalange. The cavity can be formed with sufficient space to accommodate the thickened portion. A slot can also be formed, where the slot is narrower than the thickened portion but wide enough to accommodate the thinner portion. This can allow the anchoring portions to be held securely in cavities formed in the phalanges. For example, a first cavity can be formed in a first phalange of a joint, and a second cavity can be formed in a second phalange of the joint. The anchoring portions of the flexible connector can be implanted in these cavities. The flexible central portion of the flexible connector can connect the anchoring portions together. Thus, the flexible connector can connect the phalanges. The flexible central portion of the flexible connector can act as a hinge to allow the joint to flex.

In some examples, the flexible connector can be implanted directly into bone without any separate anchor between the flexible connector and the bone. However, in other examples, anchors can be implanted into the phalanges and the flexible connector can be held by its anchoring portions in the anchors. These anchors can be made of a rigid, biocompatible material such as titanium, ceramic, surgical steel, or others. The anchors can be implanted into cavities formed in the phalanges. The anchors themselves can also have internal cavities, or cannulas, to hold the anchoring portions of the flexible connector. The anchors can also have a slot that is narrower than the thickened portion of the anchoring portions. This can allow the anchoring portions to be retained in the anchors while the flexible central portion of the flexible connector passes through the slot to connect to the other anchoring portion.

The flexible connector and anchors described herein can be configured to be implanted substantially transversely in the coronal plane. Surgical methods for implanting these devices can include making an incision on a side of a finger (i.e., on the radial aspect or ulnar aspect of the finger). Cavities can be formed in the phalanges from the side, such as by drilling bores into the bones from the side. If anchors are used, the anchors can be inserted into the bores from the side. Slots can also be cut in the phalanges to allow the flexible central portion of the flexible connector to pass from the anchoring portion in one phalange to the other. If no anchors are used, then bores can be drilled in the phalanges and slots can be cut to form a clear pathway between the bores. The flexible connector can then be pressed into the bores and slots from the side.

The flexible connector can act as a hinge to provide a full range of motion about a reference axis. In particular, the flexible central portion of the flexible connector can flex about the reference axis. In some examples, the flexible central portion can have an aspect ratio with a width significantly greater than the thickness of the flexible central portion. This can allow the flexible central portion to flex easily about the reference axis that extends across the width, while limiting flexing about other axes such as flexing about a vertical axis or a longitudinal axis. However, the flexible connector can be made of a flexible material that can allow for small amounts of flex about these other axes. In some examples, the flexible connector can also allow for rotation about an axis. The flexible connector can also be capable of stretching and compressing to mimic these capabilities in a natural joint. The flexible connector can be designed to provide a desired flexibility, extension, and compression by adjusting the elastic properties of the flexible material used to make the flexible connector, and the thickness of the flexible central portion, and other geometric features of the flexible connector.

Compared to other types of joint replacement implants, the implantable devices described herein can offer easier implantation, easier patient recovery after surgery, greater range of motion, more predictable motion outcomes, greater reliability, and other useful features. Examples showing the functions, characteristics, and arrangements of the flexible connectors and anchors are described in more detail below.

### Flexible Connectors

As mentioned above, the flexible connectors described herein include a flexible central portion, a first anchoring portion extending from a first end of the central portion, and a second anchoring portion extending from a second end of the central portion, opposite from the first end. The first anchoring portion and the second anchoring portion can each include a thickened portion located farther away from the central portion and a thinner portion, relative to the thickened portion, located nearer to the central portion.

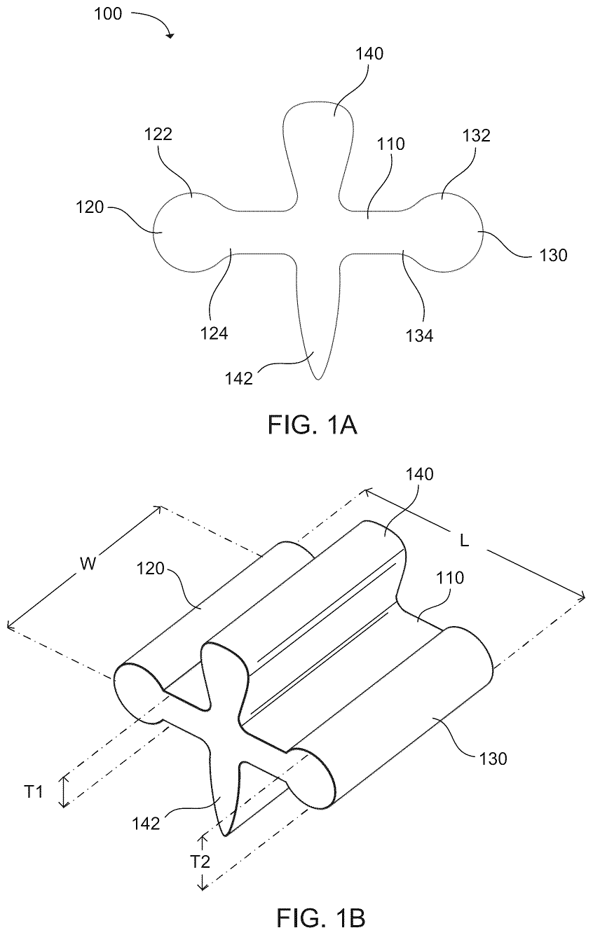

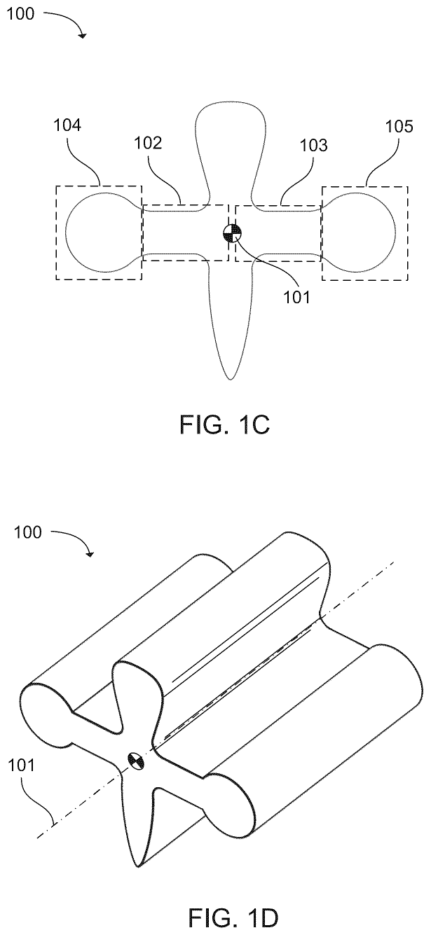

FIG. 1A shows a side-view of an example flexible connector 100 for an implantable device for arthroplasty. This flexible connector includes a flexible central portion 110, a first anchoring portion 120 extending from a first end of the central portion, and a second anchoring portion 130 extending from a second end of the central portion, opposite from the first end. The first anchoring portion includes a first thickened portion 122 and a first thinner portion 124. The second anchoring portion includes a second thickened portion 132 and a second thinner portion 134. This example also includes a dorsal bumper 140 extending from a dorsal surface of the flexible central portion and a volar bumper 142 extending from a volar (or palmar) surface of the flexible central portion. The dorsal surface of the flexible central portion is the surface facing in the direction of the back of the hand when the flexible connector is implanted in a finger joint. The volar surface faces in the direction of the palm of the hand. In FIG. 1A, the dorsal direction is upward and the volar direction is downward.

Various dimensions are described herein for the flexible connectors and the components of the flexible connectors. For example, the flexible connectors can have a length, width, and thickness. These dimensions may vary between different components of the flexible connector. For clarity, FIG. 1B shows an isometric view of an example flexible connector and several dimensions of the flexible connector are illustrated. The length (L) of the flexible connector is defined as the distance from one end of the flexible connector to the opposite end of the flexible connector. Thus, the length is measured in an end-to-end direction. This direction can also be described as the longitudinal axis of the flexible connector. The first anchoring portion 120 is at the first end of the flexible connector, and the second anchoring portion 130 is at the second end of the flexible connector. The flexible central portion 110 is defined as the central part of the connector between the first anchoring portion and the second anchoring portion. The dorsal bumper 140 and volar bumper 142 also extend from the flexible central portion in this example.

The width (W) of the flexible connector 100 is defined as the distance from one side to the opposite side. As mentioned above, FIG. 1A shows a side view of the flexible connector. As shown in FIG. 1B, this example flexible connector has a side profile that is uniform across the whole width of the flexible connector. The width direction can also be referred to as a lateral axis. In some examples, a lateral axis of the first anchoring portion 120 and a lateral axis of the second anchoring portion 130 can both lie in the coronal plane when the flexible connector is the extended position as shown (the coronal plane of the hand is defined as the plane separating the palm of the hand from the back of the hand). Additionally, the lateral axis of the first anchoring portion and the lateral axis of the second anchoring portion can be parallel. The flexible central portion 110 can also have a lateral axis that lies in the coronal plane with the lateral axes of the anchoring portions.

The thickness of the flexible central portion 110 and the anchoring portions can be measured in the top-to-bottom (dorsal to volar) direction. The flexible central portion 110 has a thickness (T1) and the second anchoring portion 130 has a thickness (T2) that is greater than the thickness of the flexible central portion. This can allow the flexible central portion to pass through a slot formed in a bone or in a separate anchor, but the thicker anchoring portion can be retained by the slot.

The dimensions of the flexible connector can be appropriate for use as a joint replacement for a finger joint, toe joint, or in some cases other joints in the body. The size of these joints can vary. For example, different fingers can have phalanges of different widths. Additionally, different patients can have fingers and toes with widely varying sizes. Therefore, the flexible connectors described herein can be provided in a variety of sizes to fit the different joints of different patients. In some examples, the flexible connector can have an overall length from about 5 mm to about 40 mm, or from about 5 mm to about 30 mm, or from about 10 mm to about 30 mm, or from about 10 mm to about 20 mm. In further examples, the flexible connector can have a width from about 5 mm to about 30 mm, or from about 5 mm to about 25 mm, or from about 5 mm to about 20 mm, or from about 10 mm to about 20 mm. The thickened portions of the anchoring portions can have a thickness from about 3 mm to about 10 mm, or from about 3 mm to about 8 mm, or from about 5 mm to about 10 mm, or from about 5 mm to about 8 mm. The thinner portion of the anchoring portions can have a thickness from about 2 mm to about 9 mm, or from about 2 mm to about 8 mm, or from about 2 mm to about 7 mm, or from about 2 mm to about 5 mm, or from about 4 mm to about 8 mm. The flexible central portion of the flexible connector can also have a thickness from about 2 mm to about 9 mm, or from about 2 mm to about 8 mm, or from about 2 mm to about 7 mm, or from about 2 mm to about 5 mm, or from about 4 mm to about 8 mm. In some examples, these sizes can be used for various finger joints of different sizes. In other examples, flexible connectors with different dimensions can be used for other joints of the body.

In another example, the flexible connector can be described in a different way. According to this description, the flexible connector can include a flexible bridge member having a first segment extending from a reference axis in a first direction and terminating at a first end. The flexible bridge member can also include a second segment extending from the reference axis in a second direction and terminating at a second end. The flexible bridge member can be configured to flex, rotate, translate, or a combination thereof, about the reference axis. The flexible connector can also include a first anchor interface located at the first end and a second anchor interface located at the second end. The first anchor interface can include a thickness greater than a thickness of the first segment of the flexible bridge member. The second anchor interface can include a thickness greater than a thickness of the second segment of the flexible bridge member.

FIG. 1C shows the various components of the flexible connector 100 according to this mode of description. The flexible bridge member includes a first segment that is the portion enclosed by dotted box 102. This first segment extends from a reference axis 101 in a first direction and terminates at a first end (i.e., the end of the dotted box). The flexible bridge member also includes a second segment enclosed in dotted box 103. The second segment extends from the reference axis and terminates at a second end. A first anchor interface is enclosed in the dotted box 104. The first anchor interface is located at the first end. The first anchor interface includes a thickness that is greater than a thickness of the first segment of the flexible bridge member. A second anchor interface is enclosed in dotted box 105. The second anchor interface is located at the second end. The second anchor interface also includes a thickness that is greater than a thickness of the second segment of the flexible bridge member.

It is noted that in some examples, the first segment or second segment of the flexible bridge member may have a thickness that varies along the length of the first and second segments. In this case, there can be at least one location along the first member where the thickness of the first member is less than the thickness of the first anchor interface. Similarly, there can be at least one location along the second member where the thickness of the second member is less than the thickness of the second anchor interface.

FIG. 1D shows an isometric view to illustrate the reference axis 101, which extends in the side-to-side (lateral) direction in the central portion of the flexible connector. It is noted that the entire flexible bridge member can flex when the device is implanted in a joint and the joint is flexed. Thus, the flexible bridge member does not bend solely at this hinge point. However, the flexing motion can be substantially about the reference axis as opposed to a vertical axis (in the dorsal to palmar direction) or a longitudinal axis.

The flexible connector 100 shown in FIGS. 1A and 1B is the same flexible connector shown in FIG. 1C and FIG. 1D. The differences in these figures are used merely to show different ways that the components and features of the flexible connector can be described. In FIG. 1A, the first anchoring portion 120 is described as having a thickened portion 122 located farther away from the flexible central portion 110, and a thinner portion 124 located nearer to the flexible central portion than the thickened portion. In contrast, in FIG. 1C, the first anchor interface 104 is described as having a thickness that is greater than a thickness of the first segment 102 of the flexible bridge member. These figures describe the same arrangement of features but use different terms. In both cases, the flexible connector includes some type of anchoring feature near the ends of the connector, where the anchoring feature has an increased thickness, and some other part nearer to the center of the connector where the thickness is less. This can allow the flexible connector to be retained by a slot formed in a bone and/or in a separate anchor part as described in more detail below.

In some examples, the flexible connector can include a flexible central portion, a first anchoring portion, and a second anchoring portion that are all integrally formed from a flexible material as a single part. Thus, the flexible central portion and the anchoring portions can comprise a contiguous flexible material. The flexible connector can be formed by a variety of processes, such as molding, extruding, machining, additive manufacturing, and others. In certain examples, the flexible connector can be formed using additive manufacturing.

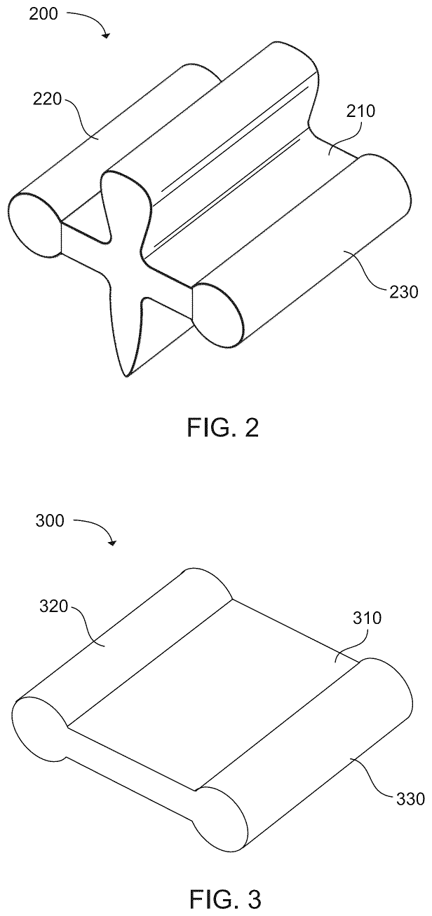

In other examples, the at least one of the first anchoring portion or the second anchoring portion can be a separate part from the flexible central portion, and the anchoring portions can be attached to the flexible central portion. FIG. 2 shows such an example flexible connector 200 that includes a flexible central portion 210, a first anchoring portion 220, and a second anchoring portion 230. The first anchoring portion and the second anchoring portion are separate components, not integrally formed with the flexible central portion as a single part. Instead, the first anchoring portion and the second anchoring portion are attached to the ends of the flexible central portion. These components can be attached using any suitable attachment method, such as adhesive, mechanical connectors, welding, or other methods. In certain examples, the anchoring portions can be made from a different material than the flexible central portion. For example, the anchoring portions can be made from a more rigid material than the flexible central portion.

The material used to form the flexible connector can be a biocompatible material. A biocompatible material can be a material that produces no immune response or a low level of immune response in the body. Biocompatible materials are sometimes also referred to as biomaterials. Flexible biocompatible materials can include polymers, flexible metals, woven metal meshes, and others. In one example, the polymer can be a silicone polymer. Other examples can include medical grade plastic, biocompatible elastic polymers, polyetheretherketone (PEEK), polypropylene (PE), ultra-high molecular weight polyethylene (UHMWPE), polypropylene (PP), polytetrafluoroethylene (PTFE), perfluoroalkoxy (PFA), fluorinated ethylene propylene (FEP), polyurethane (PU), thermoplastic polyurethane (TPU) or others.

The examples shown above include a dorsal bumper extending from a dorsal surface of the flexible connector and a volar bumper extending from a volar surface of the flexible connector. These can be useful for cushioning the bones on either side of the implant and preventing direction bone-on-bone contact. However, the flexible connector can also be made without these bumpers. FIG. 3 shows an example flexible connector 300 that does not include a volar bumper or a dorsal bumper. This flexible connector includes a flexible central portion 310 that has a flattened rectangular shape, with a uniform thickness across the width of the flexible central portion and along the length of the flexible central portion. A first anchoring portion 320 extends from a first end of the central portion, and a second anchoring portion 330 extends from a second end of the central portion. This flexible connector can operate as the previous examples, in that the anchoring portions can be anchored in bone cavities or in cannulas of separate anchor parts, and the flexible central portion can flex to provide a range of motion to the joint.

Because the flexible connector can be subjected to repeated flexing and stretching motions, it can be useful to design the flexible connector to withstand the stresses from these motions for as long as possible without failing. Stretching and flexing can induce stresses in the flexible connector that may tend to tear the flexible connector and propagate tears that have already formed. If stresses are concentrated in certain locations in the flexible connector, then these locations can become likely failure points. Therefore, the flexible connector can be designed to spread the stresses out throughout the flexible connector instead of concentration the stresses at specific locations. One type of potential failure point may occur at sharp concave edges or corners. As used herein, “concave edge” refers to an edge where two faces meet at a sharp transition, and where the faces and the transition together form a concave surface. In contrast, a convex edge would be an edge where two face meet to form a convex surface, such as the edges of a cube. Concave edges can tend to concentrate stresses from stretching and flexing more than convex edges. Therefore, it can be useful to design the shape of the flexible connector to have few or no concave edges. In some examples, the entire flexible connector can be devoid of concave edges. In other examples, the flexible connector can be designed so that the anchoring portions transition smoothly to the flexible central portion without a concave edge. In other words, there is no sharp edge between the thicker anchoring portion and the thinner flexible central portion. At the transition between the anchoring portion and the flexible central portion, there can be a rounded or filleted edge in some examples. Similarly, the transition between the flexible central portion and the dorsal bumper or volar bumper can also be designed without concave edges. In certain examples, the bumpers can join the flexible central portion at curved or filleted edges.

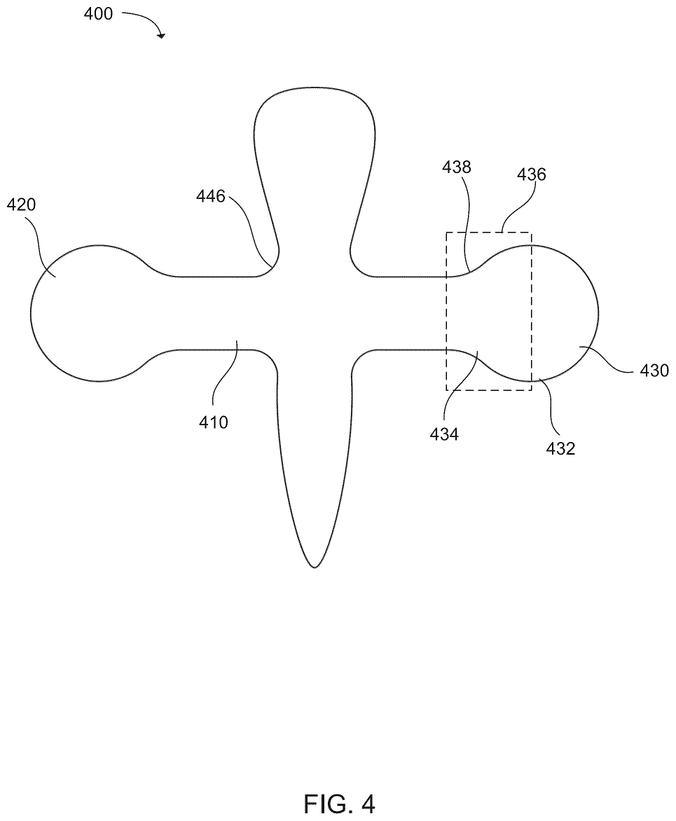

FIG. 4 shows a side view of an example flexible connector 400 that includes such rounded edges. This example includes smooth transitions between the flexible central portion 410 and the first anchoring portion 420 and the second anchoring portion 430. The smooth transition can be described as a transition portion 436 (indicated by the dashed box) of the anchoring portion. As shown in this figure, the second anchoring portion includes a transition portion that extends from the thickened portion 432 of the second anchoring portion to the flexible central portion. This transition portion includes the thinner portion 434 of the second anchoring portion. The transition portion does not have any concave edges, and the second anchoring portion does not join the flexible central portion at a concave edge. Instead, the second anchoring portion transitions to the flexible central portion through a curved surface 438. The round shape of the anchoring portions combined with the round curved surface of the transition portions gives the anchoring portions a teardrop-shaped profile. The teardrop-shaped profile transitions smoothly to the flexible central portion.

In some examples, the curved surface of the transition portion can have a radius of curvature from about 0.1 mm to about 10 mm, or from about 0.5 mm to about 10 mm, or from about 1 mm to about 10 mm, or from about 1 mm to about 5 mm. In further examples, edges or corners that have a radius of curvature less than 0.1 mm can be considered “sharp” edges or corners. In other examples, the bumpers can also have transition portions that include a curves surface with a similar radius of curvature.

As mentioned above, in some examples the various features and components of the flexible connector can have a thickness that is uniform across the entire width of the flexible connector. For example, the anchoring portions in the previous examples have a somewhat cylindrical shape that has the same thickness and cross-section across the entire width of the flexible connector. However, in other examples, the thickness can vary across the width of the flexible connector. The thickness of the anchoring portions, the flexible central portions, the bumpers, and other components of the flexible connector can vary at different locations across the width of the flexible connector.

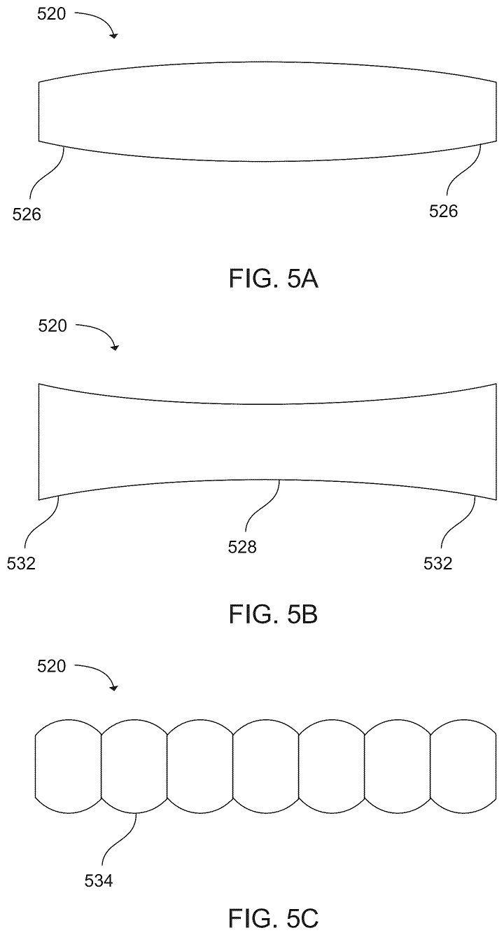

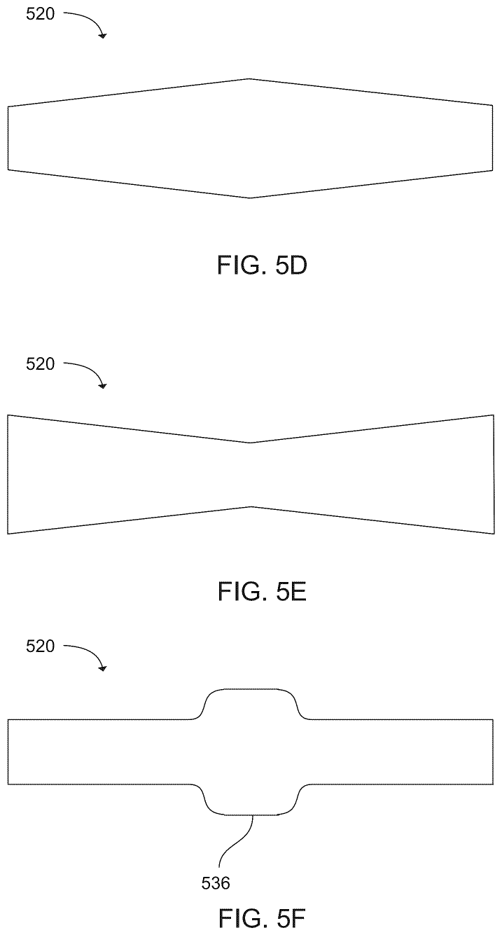

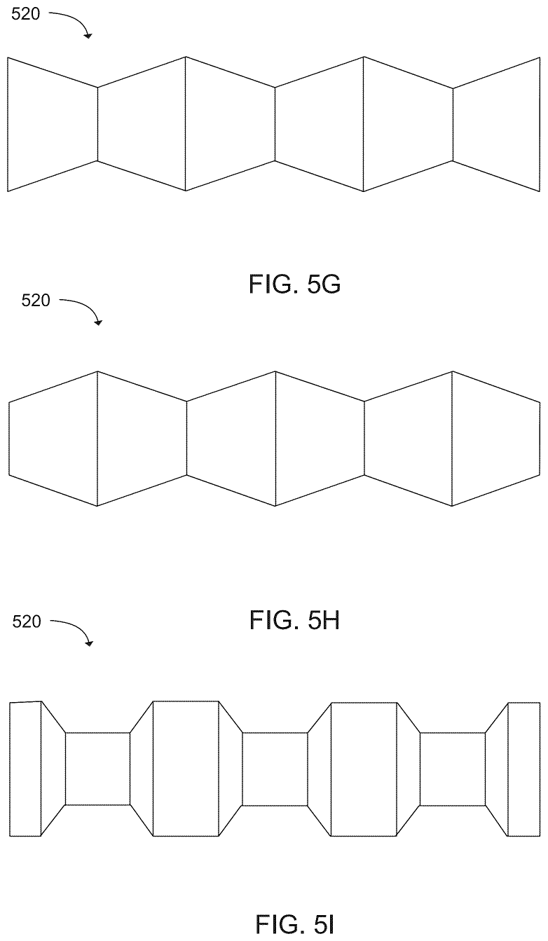

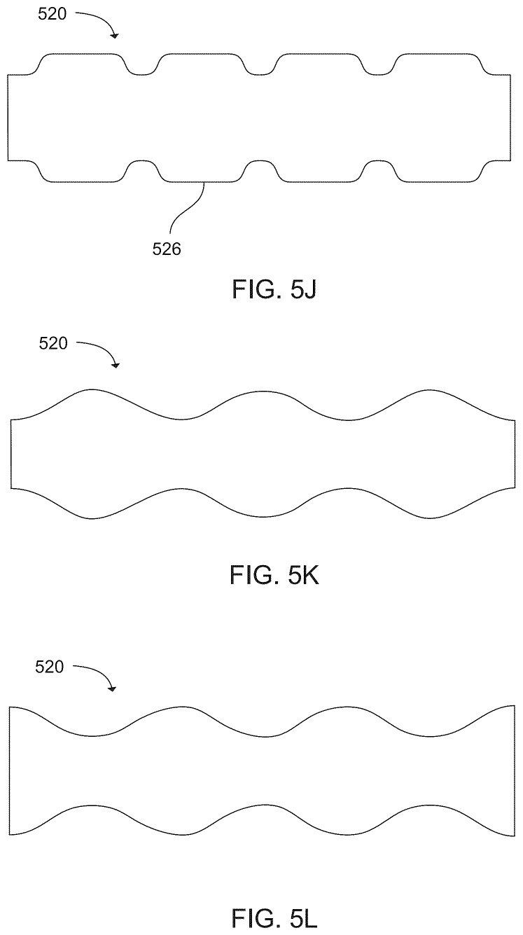

In certain examples, the anchoring portions can include a taper on one side or both sides. The taper can be referred to as a lateral taper because it is located at a side of the flexible connector. FIG. 5A shows an end-on view of an anchoring portion 520 that has lateral tapers 526 on both sides. The tapers can be useful to make it easier to press the flexible connector laterally into a bore formed in a bone, or into a cannula in a hollow anchor. Additionally, in some examples this anchoring portion can be inserted into a cavity that has a similar shape that conforms to the shape of the tapered anchoring portion. In other words, the cavity can also have a taper on either side, so that the cavity is narrow on the sides and thicker in the middle. This can help retain the anchoring portion in the cavity. The tapered sides are one example of a non-uniform thickness profile because the thickness of the anchoring portion is not uniform across the width of the flexible connector. In other examples, the anchoring portions can have a variety of other non-uniform thickness profiles. These can be useful for facilitating retention of the anchoring portions inside anchors that have a cavity of the same shape that conforms to the non-uniform thickness profile.

FIG. 5B shows another end view of an anchoring portion 520 with a non-uniform thickness profile. This non-uniform thickness profile includes a thinner middle portion 528 and thicker side portions 532.

FIG. 5C shows another example anchoring portion 520 with a non-uniform thickness profile. This non-uniform thickness profile includes a series of rounded bumps 534.

FIG. 5D shows another example anchoring portion 520 with a non-uniform thickness profile. This profile slopes from thinner sides to a thicker centerline.

FIG. 5E shows another example anchoring portion 520 with a non-uniform thickness profile. This profile slopes from thicker sides to a thinner centerline.

FIG. 5F shows another example anchoring portion 520 with a non-uniform thickness profile. This profile includes a rounded thickened region 536 at the middle of the anchoring portion.

FIG. 5G shows another example anchoring portion 520 with a non-uniform thickness profile. This profile includes a repeated series of slopes from thick to thinner and back to thicker again.

FIG. 5H shows another example anchoring portion 520 with a non-uniform thickness profile. This profile includes a repeated series of slopes from thin to thicker and back to thin again.

FIG. 5I shows another example anchoring portion 520 with a non-uniform thickness profile. This profile includes a repeated series of segments having a first uniform thickness alternating with segments having a second greater uniform thickness, where the thicker segments are connected to the thinner segments by sloped portions.

FIG. 5J shows another example anchoring portion 520 with a non-uniform thickness profile. The profile includes a repeated series of rounded thickened region 536.

FIG. 5K shows another example anchoring portion 520 with a non-uniform thickness profile. This profile starts thinner at the sides and has a repeated wave pattern across the width of the anchoring portion.

FIG. 5L shows another example anchoring portion 520 with a non-uniform thickness profile. This profile has a similar wave pattern to FIG. 5K but starting at a thicker thickness at the sides. Any of the above non-uniform thickness profiles can be used together with an anchor that has a cavity shaped to conform to the non-uniform thickness profile. The flexible connector can be made from a flexible material that can be compressed enough to allow the anchoring portion to squeeze into the conforming cavity, even though the non-uniform thickness profile can include thicker parts that will be squeezed when pressing through thinner parts in the cavity.

The flexible connectors can be used together with fixation rods in some examples. The anchoring portions of the flexible connector can include rod sleeves to accommodate the fixation rods. The rod sleeve can be an empty space within the anchoring portion of the flexible connector that is shaped and size to accommodate a fixation rod. The rod sleeve can extend along a width direction of the flexible connector (i.e., parallel to the lateral axis). The rod sleeve can be open on one or both ends in some examples, so that a fixation rod can be inserted into the rod sleeve. Inserting a fixation rod can be useful in some cases to expand the anchoring portion, giving the anchoring portion a greater thickness, or to make the anchoring portion more rigid, or to provide an additional feature such as an end cap integrated in the fixation rod, or a combination of these.

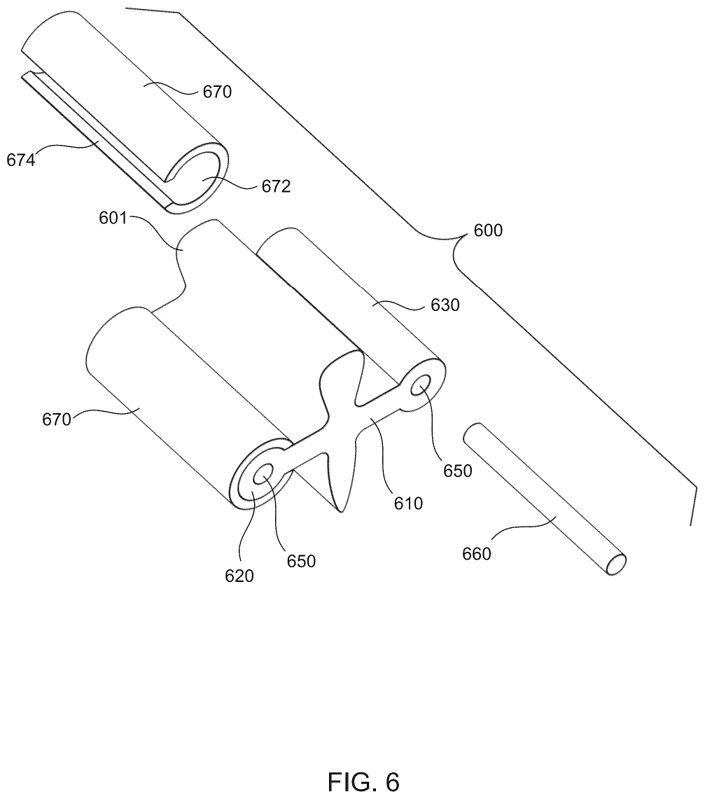

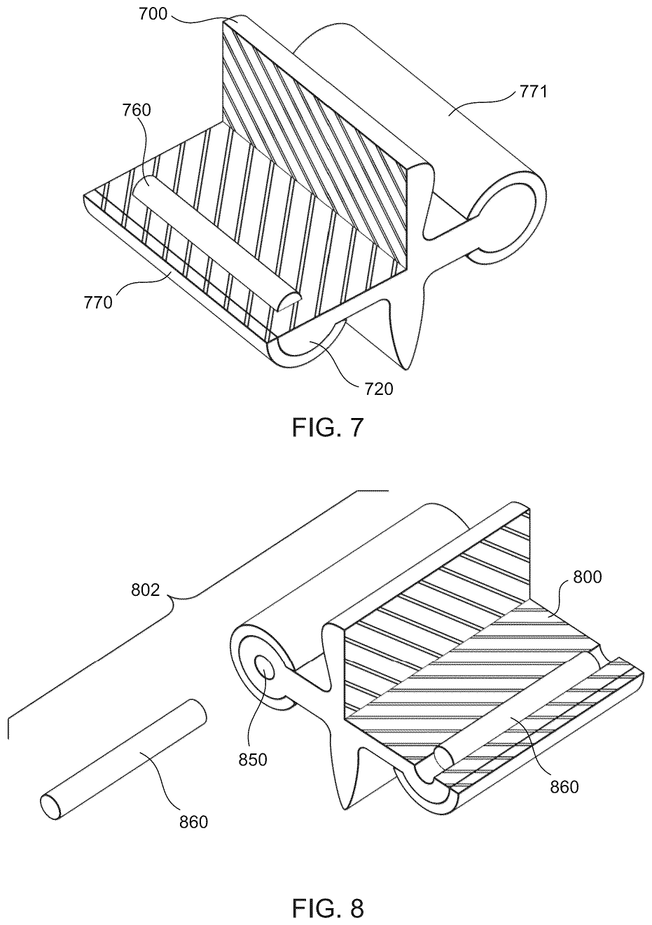

FIG. 6 shows an exploded view of an example stemless implantable device for arthroplasty 600. This example includes a flexible connector 601 that has rod sleeves 650 formed in the first anchoring portion 620 and the second anchoring portion 630. Fixation rods 660 can be inserted into these rod sleeves. This example also includes two anchors 670 that include an interior cannula 672 and a slot 674. The anchoring portions of the flexible connector can be pressed into the interior cannula of the anchors, and the flexible central portion 610 of the flexible connector can pass through the slots.

In some examples, the fixation rods can be embedded in the anchoring portions as integral parts of the flexible connector. For example, the flexible connector can be molded with the rod sleeves inside the anchoring portions, or formed by additive manufacturing with the rod sleeves inside the anchoring portions at the time of manufacture. FIG. 7 shows a partial cutaway view of an example flexible connector 700 that has a fixation rod 760 integrally embedded in the first anchoring portion 720. The space taken up by the fixation rod can still be referred to as a rod sleeve, although the rod sleeve was not formed as an empty space for the rod be inserted later. This figure also shows part of a first anchor 770 and a second anchor 771.

In other examples, the rod sleeves can be open at one end or both ends. FIG. 8 shows an example flexible connector 800 that has rod sleeves 850 formed with openings at both ends of the rod sleeves. It is noted that the “ends” of the rod sleeves are actually located on the sides of the flexible connector because the rod sleeves extend from side to side across the anchoring portions of the flexible connector. A fixation rod 860 is shown inserted into one rod sleeve. This fixation rod has a length that is less than the width of the anchor portion. Thus, the fixation rod does not extend all the way across the anchor portion. In other examples, the fixation rod can have a length that allows the fixation rod to extend all the way across the anchor portion or all the way into the rod sleeve.

The fixation rods can be smooth, cylindrical rods in some examples. In other examples, the fixation rods can have surface features to facilitate retention of the fixation rods in rod sleeves. In some examples, fixation rods can have surfaces features such as threads, annular ribs, barbs, a smooth surface, or a combination thereof. In further examples, the fixation rods can have a non-uniform thickness profile. The rod sleeves can also have a cavity shaped to conform to the non-uniform thickness profile.

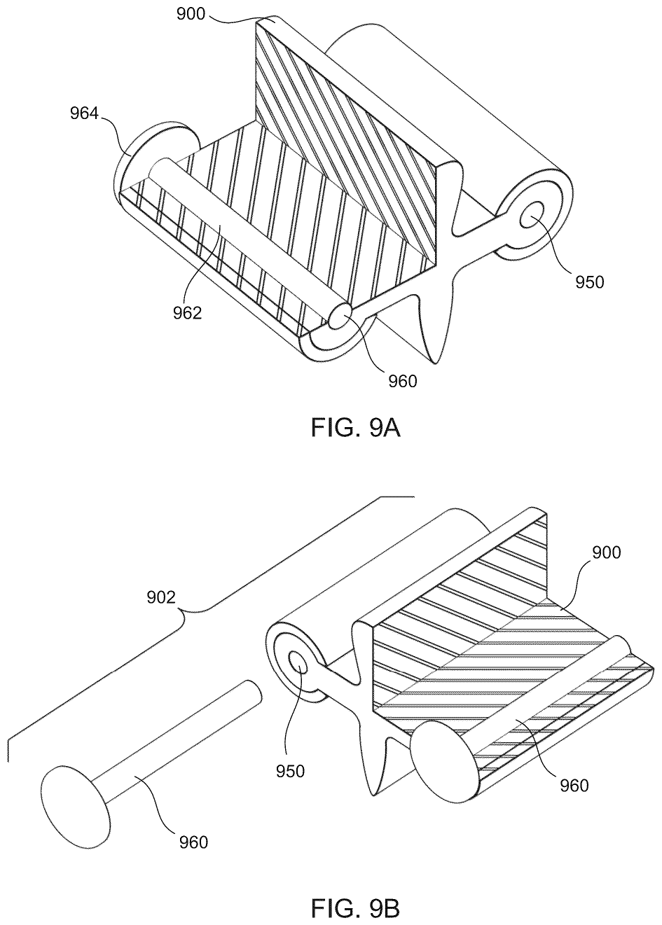

FIG. 9A shows an example flexible connector 900 with rod sleeves 950. A fixation rod 960 is inserted into one rod sleeve. In this example, the fixation rod includes an insertion portion 962 that is inserted into the rod sleeve and an end cap portion 964 at the end of the fixation rod. The end cap portion has a diameter greater than the diameter of the insertion portion. Therefore, the end cap portion does not enter into the rod sleeve, but abuts the side if the flexible connector just outside the rod sleeve. FIG. 9B shows a second fixation rod being inserted into the flexible connector. These parts together can make an implantable device 902.

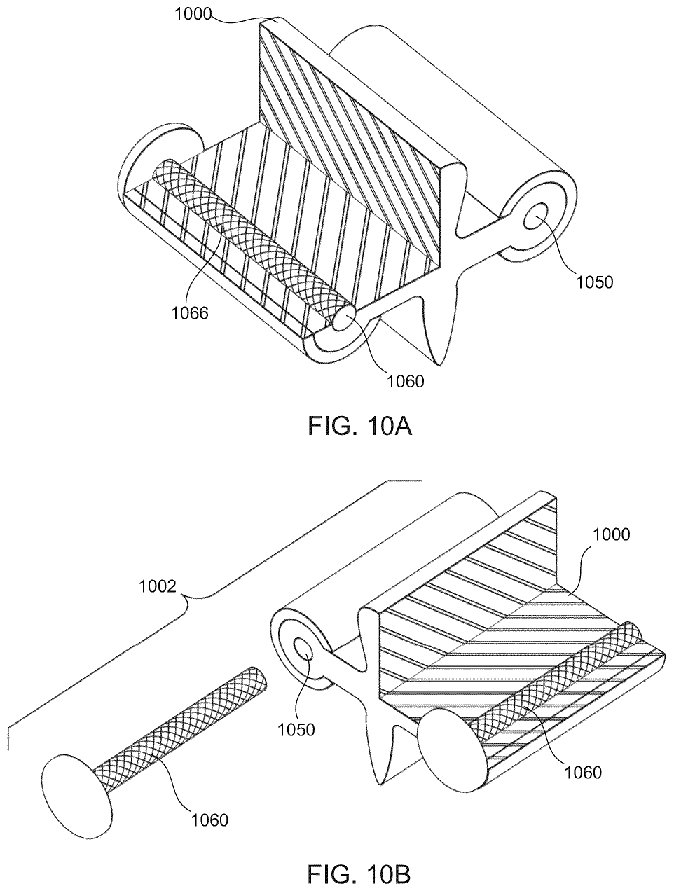

FIG. 10A shows another example flexible connector 1000 with rod sleeves 1050. As in the previous example, a fixation rod 1060 is inserted into one of the rod sleeves. In this example, the fixation rod has surface grooves 1066 that can facilitate retention of the fixation rod in the rod sleeve by increasing friction between the fixation rod and the rod sleeve. FIG. 10B shows the flexible connector as a second fixation rod is about to be inserted into the rod sleeve. These parts together can make up an implantable device 1002.

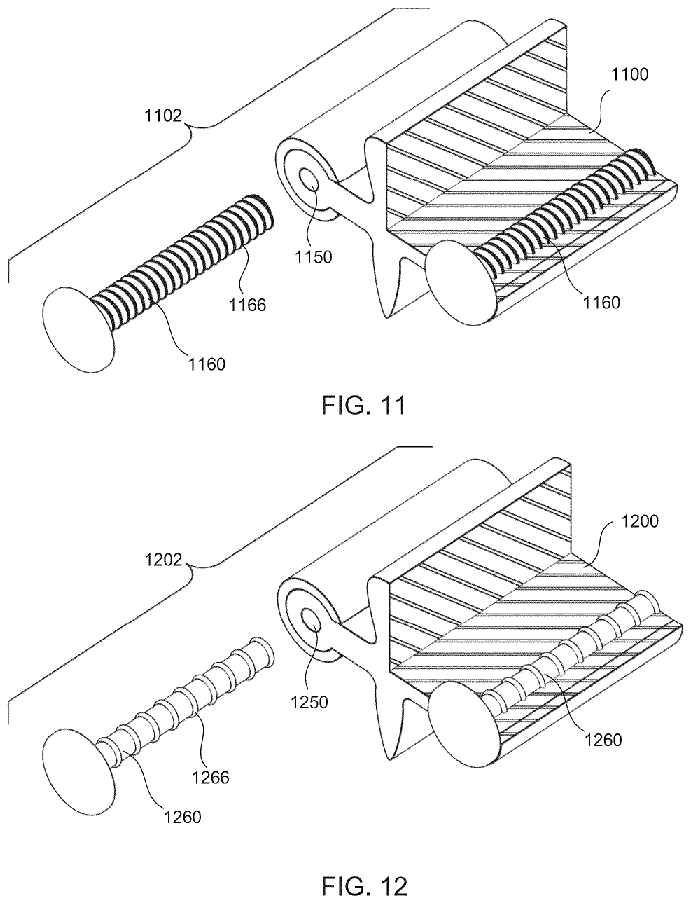

FIG. 11 shows another similar example flexible connector 1100. In this example, the fixation rod 1160 has surface threads 1166 that can allow the fixation rod to be screwed into the rod sleeves 1150. The rod sleeves can be formed with a similar thread pattern that conforms to the threads of the fixation rod. These parts together can make up an implantable device 1102.

FIG. 12 shows another example flexible connector 1200. In this example, the fixation rod 1260 includes annular ribs 1266 spaced along the rod. The rod sleeves 1250 can be formed with spaces to accommodate these ribs when the fixation rod is inserted. These parts together can make up an implantable device 1202.

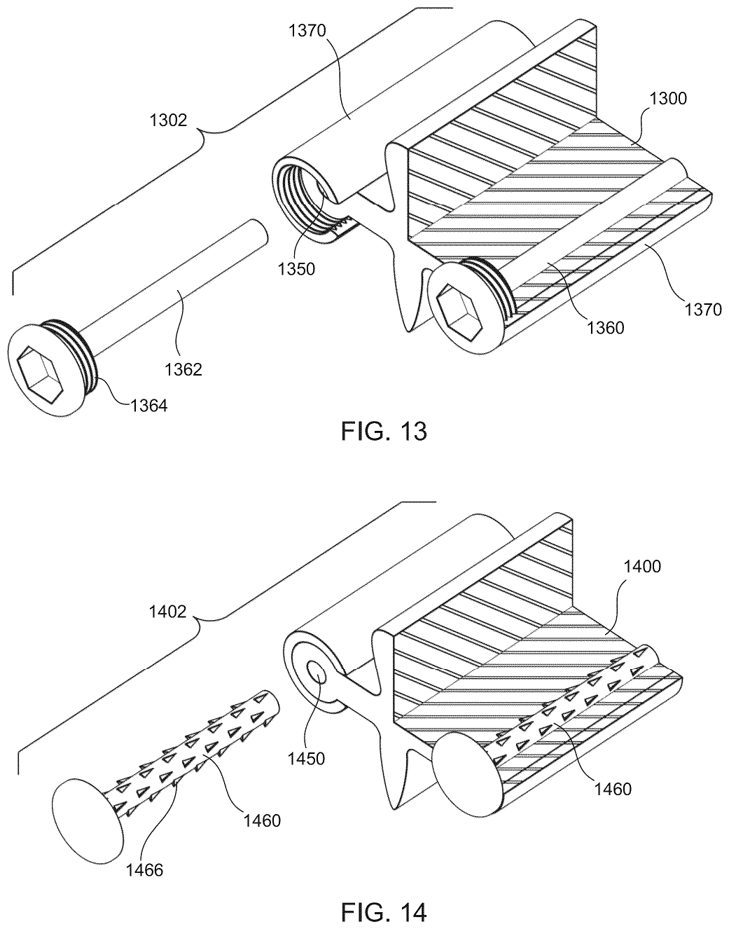

FIG. 13 shows another example flexible connector 1300 with rod sleeves 1350. I this example, the fixation rod has an end cap portion 1364 and an insertion portion 1362. The insertion portion has a smooth surface, but the end cap portion has surface threads 1366. The anchor 1370 also has an internally threaded opening so that the threaded end cap can screw into the anchor. Thus, the fixation rod can be retained by the threaded connection between the end cap and the anchor. These parts together can make up an implantable device 1302.

FIG. 14 shows another example flexible connector 1400. In this example, a fixation rod 1460 with surface barbs 1466 is inserted into a rod sleeve 1450. The surface barbs can be slanted away from the insertion end of the fixation rod to allow the fixation rod to be pressed into the rod sleeve. However, the barbs can catch the rod sleeve and prevent the fixation rod from being pulled back out after it has been inserted. These parts together can make up an implantable device 1402.

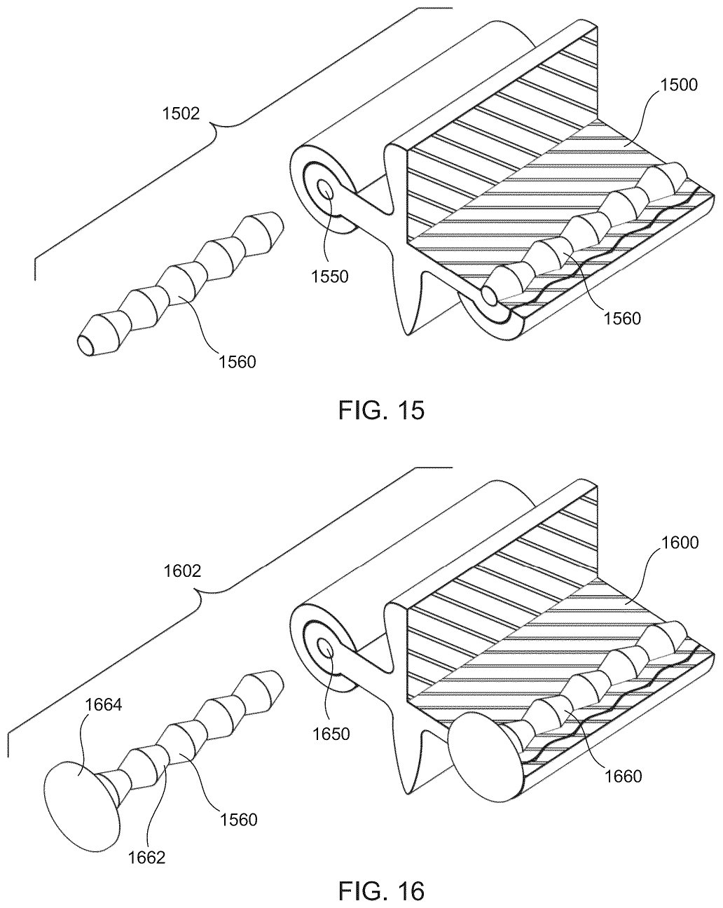

FIG. 15 shows an example flexible connector 1500 with a fixation rod 1560 inserted in a rod sleeve 1550. In this example, the fixation rod has a non-uniform thickness profile with a repeated series of sloping portions. The fixation rod starts at a smaller diameter and the slopes to a larger diameter, and then slopes back down to the smaller diameter and this pattern repeats multiple times along the length of the fixation rod. The rod sleeve is formed as a cavity having a shaped that conforms to the non-uniform thickness profile of the fixation rod. This can facilitate retention of the fixation rod in the correct location in the rod sleeve after the fixation rod has been inserted. These parts together can make up an implantable device 1502. FIG. 16 shows a similar example flexible connector 1600 where the fixation rod 1660 has an end cap portion 1664 and an insertion portion 1662 with the same non-uniform thickness profile. These parts together can make up an implantable device 1602.

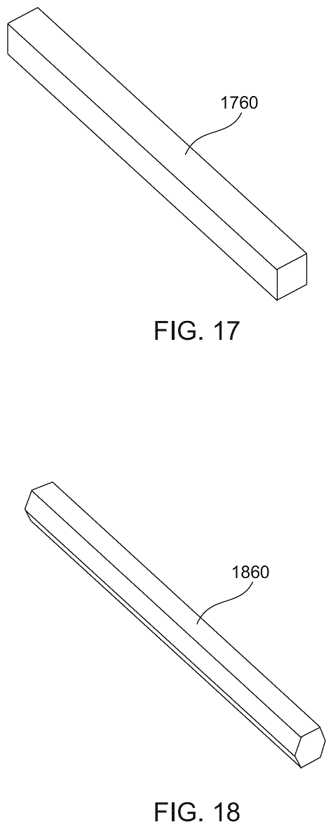

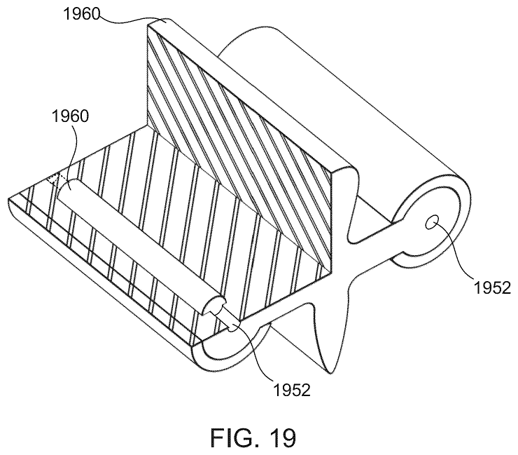

The fixation rods shown in the examples have a cylindrical shape or a circular cross section. However, fixation rods may have other cross-section shapes. For example, the fixation rods can have a polygonal cross-section, a triangular cross-section, a square cross-section, a hexagonal cross-section, a rectangular cross-section, an elliptical cross-section, a cross-shaped cross-section, or another shaped cross-section. FIG. 17 shows an example fixation rod 1760 that has a square cross-section. FIG. 18 shows another example fixation rod 1860 that has a hexagonal cross-section.

In further examples, the rod sleeves can include a cavity shaped to retain a fixation rod and an entrance portion with a smaller diameter. FIG. 19 shows one such example flexible connector 1900. In this example, the rod sleeves are open on end and closed on the other end. Each rod sleeve includes an entrance portion 1952 at the opening of the rod sleeve. The entrance portion has a diameter that is less than the diameter of the fixation rod 1960. However, the fixation rod can be pressed through the entrance portion because the flexible material of the flexible connector can stretch and compress. Inside the entrance portion, the rod sleeve opens out into a larger diameter retention portion that substantially matches the diameter of the fixation rod. Thus, when the fixation rod is pushed in far enough, it can be retained in the retention portion and the entrance portion can contract back to its smaller diameter, holding the fixation rod in place. In further examples, the rod sleeve can include an exit portion oppose from the entrance portion. The exit portion can also have a smaller diameter than the fixation rod. The dotted lines in this figure show the location of an exit portion that can be opposite the entrance portion in other examples.

As mentioned above, the width of the flexible connector can be the dimension in the side-to-side direction. In some examples, the flexible connector can have a uniform width all the way along the length of the flexible connector. However, in other examples, the width can vary in different locations along the length of the flexible connector. In some examples, the flexible connector can be wider in a central portion than at the ends. In a particular example, the width of the central flexible portion can be a first width adjacent to at least one of the anchoring portions, and then the width can increase to a greater second width at a location between the anchoring portions.

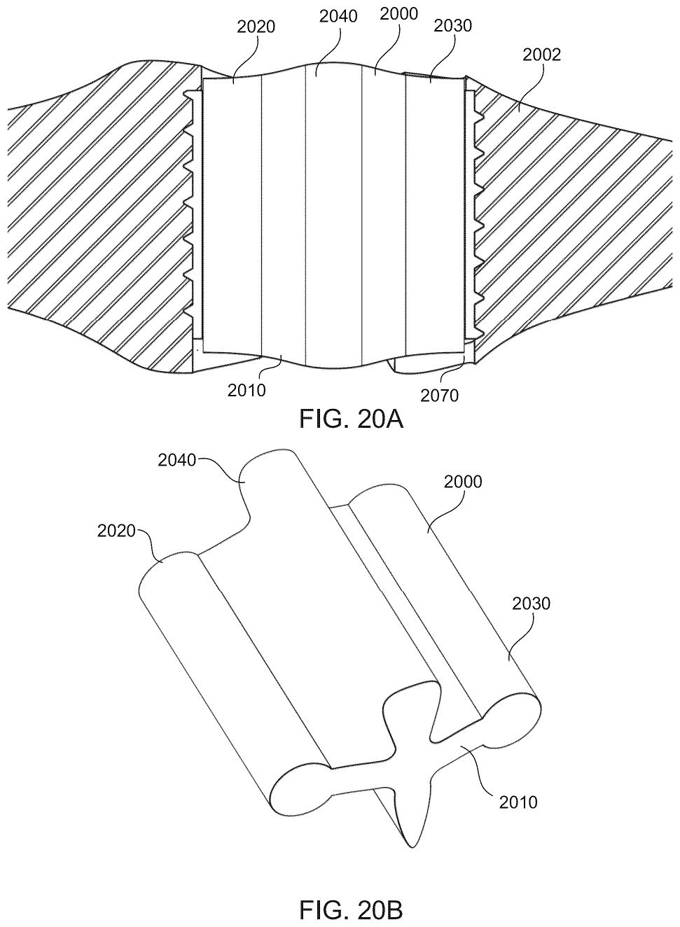

FIG. 20A shows a top-down view of an example flexible connector 2000. This flexible connector has a width that varies along the length of the flexible connector. Additionally, this figures shows a partial cross-section of anchors 2070 and bones 2002 that the anchors have been implanted into. In this example, the flexible connector includes a first anchoring portion 2020 and a second anchoring portion 2030. The anchoring portions are held in the anchors. The flexible connector also includes a flexible central portion 2010 that connects the anchoring portions. The top-down view also shows a dorsal bumper 2040 extend upward from the central portion. As shown in this figure, the central portion has a width that increases from a first width adjacent the anchoring portions to a greater second width at the middle of the central portion. This design can be useful because the anchoring portions can have a width that is smaller than the full width of the bones, which can help fit the anchoring portions in the anchors that are implanted in the bones. However, it can also be useful to have the central portion of the flexible connector occupy the entire width of the joint capsule, or approximately the whole width of the bones of the joint, to provide stability to the joint. The increased width can also allow for the bumpers to have an increased width so that the bumpers can prevent contact between the bones across the whole width of the bones. FIG. 20B is an isometric view showing the flexible connector, to show how the flexible connector bulges to a greater width in the central portion.f

As mentioned above, in some examples the flexible connector can be configured to stretch in the longitudinal direction to more closely approximate a natural arc of motion compared to a simple hinge. The stretching ability can depend on the elasticity of the material used to form the flexible connector and on the geometry of the flexible connector. In some examples, the flexible connector can be designed to stretch to a maximum extension from about 1% to about 25% longer than an unextended length. In other examples, the maximum extension can be from 1% to 15%, or from about 1% to 10%, or from 1% to 5%, or from 5% to 25%, or from 5% to 15% longer than the unextended length.

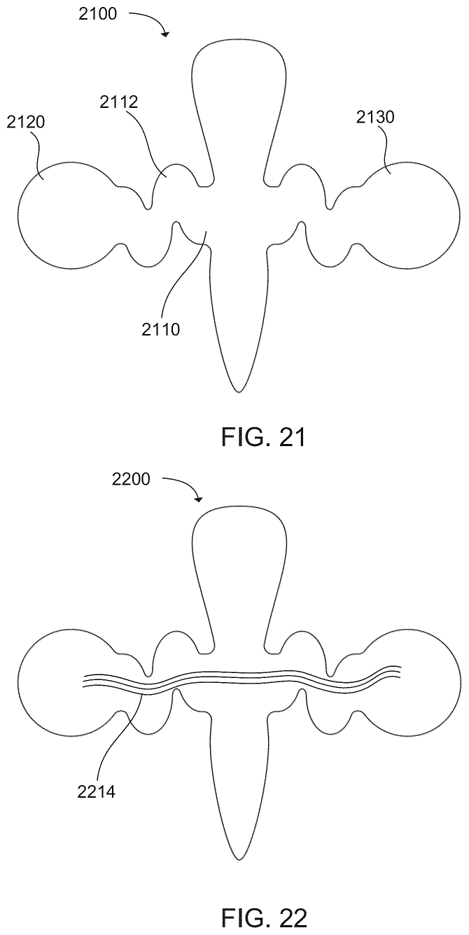

In some examples, the flexible connector can be configured with a wave-shaped portion to facilitate stretching of the flexible central portion along the longitudinal axis of the flexible connector. FIG. 21 shows a side view of an example flexible connector 2100 with this design. The flexible central portion 2110 includes a wave-shaped portion 2112. This wave shape can increase the stretching capability and maximum extension of the flexible connector. The wave shape in this figure includes one upward wave started proximate to the center of the flexible connector, and one downward wave farther from the center of the flexible connector, and then the flexible center portion returns to its normal height before transitioning to the first anchoring portion 2120 and second anchoring portion 2130 on either end of the flexible connector. In other examples, a greater number of waves can be included, with multiple upward and downward waves in series between the center of the flexible connector and the anchoring portions.

The maximum extension of the flexible connector can also be controlled using fibers in some examples. A plurality of fibers can extend along a longitudinal axis of the flexible connector, and the fibers can define a maximum extension of the flexible connector. In some examples, the fibers can be stretchable, but the fibers can stretch to a maximum extension and then stop stretching. Alternately, the fibers can be non-stretchable fibers. The fibers can be arranged with some slack in the fibers when the flexible connector is in a non-extended state. When the flexible connector stretches and extends to the point that the slack is taken out of the fibers, then the fibers can prevent additional stretching of the flexible connector. FIG. 22 shows an example flexible connector 2200 that includes a plurality of fibers 2214 extending along the longitudinal axis of the flexible connector. These fibers are shown having a certain amount of slack. This allow the flexible connector to stretch until there is no more slack in the fibers and the fibers can prevent further extension. In some examples, the fibers can be embedded within the flexible connector. In other examples, the fibers can be adhered to a surface of the flexible connector by adhesive or by another attachment method. In various examples, the fibers can be stretchable fibers or non-stretchable fibers. Non-stretchable fibers can be used to define a maximum extension of the flexible connector. In certain examples, the flexible connector can be designed to be at its maximum extension when relaxes, i.e., the flexible connector is not capable of stretching. In such examples, taut non-stretchable fibers can be included in the flexible connector to prevent the flexible connector from stretching. In further examples, both stretchable and non-stretchable fibers can be used to reinforce the flexible connector.

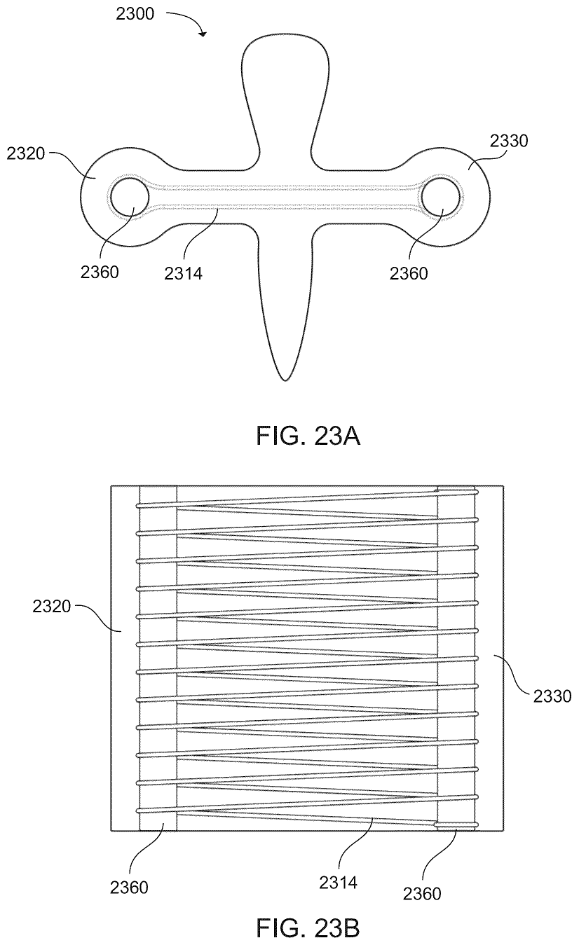

FIG. 23A shows a cross-sectional side view of another example flexible connector 2300 that has embedded fibers 2314. This example also includes fixation rods 2360 in the first anchoring portion 2320 and the second anchoring portion 2330. The fibers wrap around the fixation rods in this example. FIG. 23B shows a cutaway top down view of this example, illustrating how the plurality of fibers wrap around and extend between the fixation rods. In other examples, the fibers can be attached to fixation rods by other attachment methods besides wrapping.

Regarding the dorsal and volar bumpers, it can be useful to include dorsal and volar bumpers extending from the cesntral portion of the flexible connector. The bumpers can cushion the bones on either side of the joint and prevent the bones from contacting one another. In particular, the dorsal bumper can prevent the bones from contacting when the joint is extended, and the volar bumper can prevent the bones from contacting when the joint is flexed. Additionally, the bumpers can provide stability to the joint. In some examples, the dorsal bumper can help prevent hyperextension of the joint.

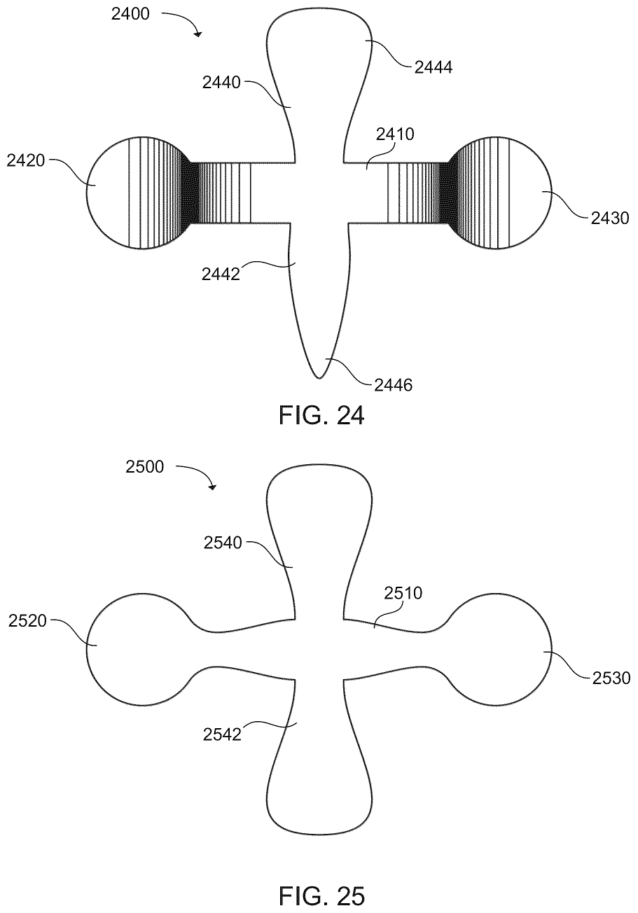

In certain examples, the dorsal bumper can have an upper bulged portion to prevent contact between a proximal phalange and a distal phalange when the flexible connector is implanted. FIG. 24 shows an example flexible connector 2400 with a dorsal bumper 2440 and a volar bumper 2442. The dorsal bumper includes a bulged upper portion 2444 that bulges to a greater dimension compared to a location lower on the bumper, closer to the flexible central portion 2410 of the flexible connector. The volar bumper tapers to a lower narrowed portion 2446. This can facilitate flexion of the joint into which the flexible connector is to be implanted. The tapered narrow portion can allow the bones to get closer together when the joint is flexed, providing a better range of motion and more natural feeling motion. This example also has a gradient of durometer hardness over the length of the flexible connector, which is represented by the vertical black line gradient. In this example, a first stiffness is present at the transition between the first anchoring portion 2420 and the flexible central portion, as well as between the second anchoring portion 2430 and the flexible central portion. Because these transitions are locations where stress may be concentrated, increasing the hardness or stiffness of the flexible connector in these locations can make the flexible connector more durable and reliable. The hardness or stiffness can be represented by Shore durometer hardness in some examples. Varying the durometer can also allow rotation within the anchor and simultaneous stretching or flexing within the central portion of the flexible connector when a uniform force is applied in flexion through the implant.

In a different example, the flexible central portion can have a varying durometer hardness along the length of the flexible central portion. A first, greater stiffness can be present at the location where the dorsal and volar bumpers extend from the flexible central portion. A second, lower stiffness can be present farther from dorsal and volar bumpers.

Another way to strengthen parts of the flexible connector that may be subject to stress concentration is to make those parts thicker. In some example the flexible central portion can have a vary thickness over a length of the flexible central portion. The thickness adjacent the bumpers can be greater than the thickness at another location farther away from the bumpers. FIG. 25 shows an example flexible connector 2500 that has a flexible central portion 2510 with a greater thickness adjacent to the dorsal bumper 2540 and the volar bumper 2542 and a smaller thickness adjacent to the first anchoring portion 2520 and the second anchoring portion 2530. This example has a bulged dorsal bumper and a bulged volar bumper instead of the tapered volar bumper shown in the previous example. It is noted that a variety of other shapes can also be used for the bumpers.

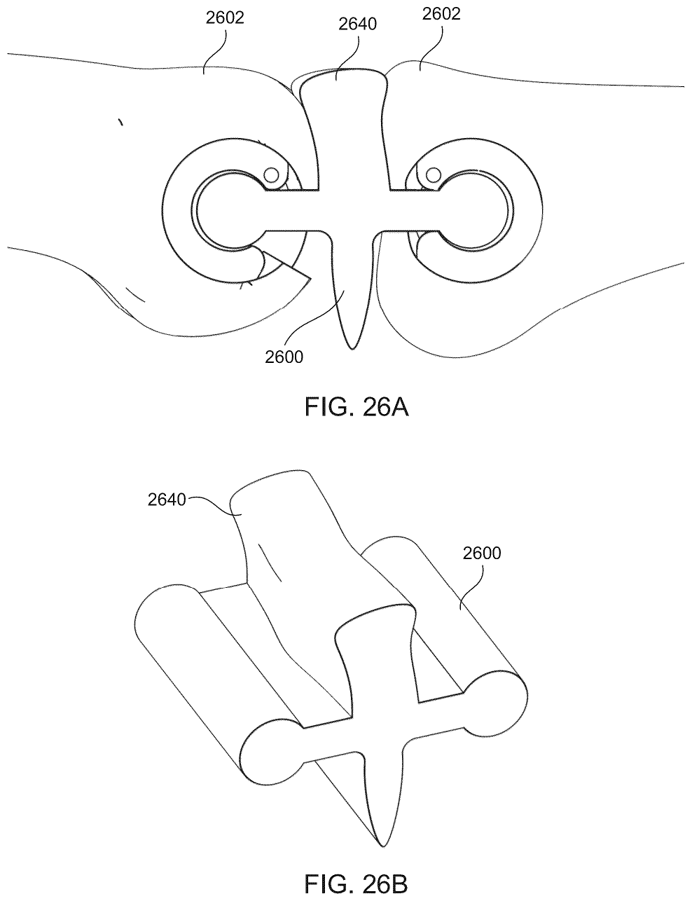

In some examples, at least one of the bumpers can be shaped to conform to the surfaces of the bones on either side of the joint. For example, the dorsal bumper can be shaped to fit and conform with the surface of a proximal phalange and a distal phalange where the surfaces contact the dorsal bumper when the joint is extended. This can be accomplished, in some cases, by preforming the bumper to have a complimentary surface shape to fit together with the surface shapes of the bones. In other examples, the bumper can be made of a strain rate-sensitive material that resist rapid strains while allowing slower strains, similar to memory foam. These materials can conform to the surface shape of the bones and then at least partially retain the conforming shape. FIG. 26A is a side view showing a flexible connector 2600 implanted into two phalanges 2602. The flexible connector has a dorsal bumper 2640 that conforms to the shape of the bones on either side of the joint when the joint is extended. In some cases, using bumpers that conform to the shape of the bones in this way can add more lateral stability to the joint. FIG. 26B shows an isometric view of the flexible connector alone.

The examples shown above have include bumpers formed as an integral part of the flexible connector. In certain examples, the dorsal bumper and volar bumper can be integrally formed with the flexible central portion of the central connector as a single part. However, in other examples, the bumpers can be formed as one or more separate parts that are attached to the flexible central portion of the flexible connector. In certain examples, the dorsal bumper and volar bumper can be formed as a single unit that is a separate part from the flexible central portion of the flexible connector, but which can be attached to the flexible central portion. The separate part comprising the bumpers can include an opening of an appropriate size for the flexible central portion of the flexible connector to pass through. In some examples, at least one of the flexible central portion or the separate part comprising the bumpers can include a surface alignment feature to facilitate alignment of the separate part with the flexible central portion. For example, one part can have bumps and the other part can have indentations to match the bumps to facilitate alignment and retention of the parts. In another example, one part can include ridges configured to lock the other part in place after assembly.

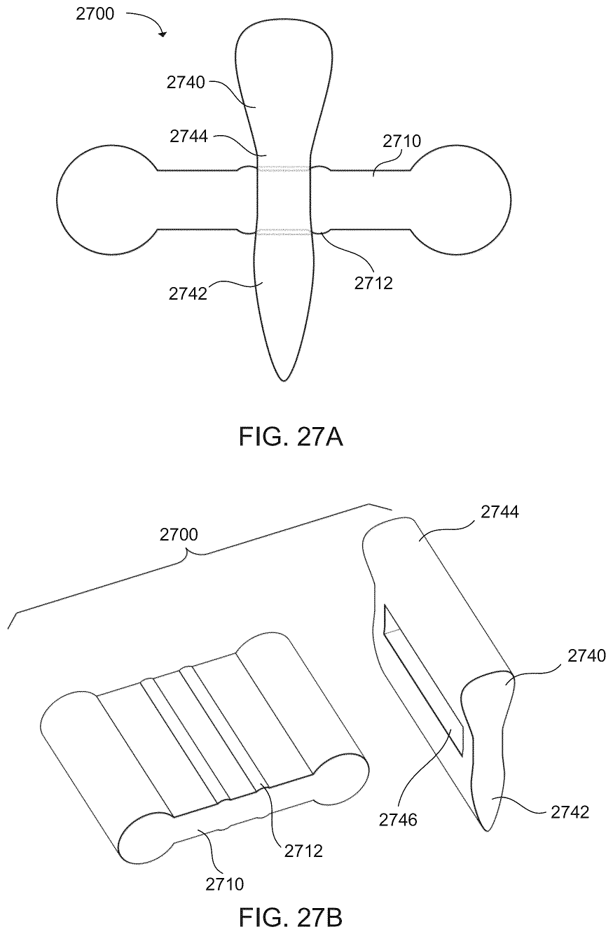

FIG. 27A shows a cross-sectional side view of an example flexible connector 2700 that includes a dorsal bumper 2740 and a volar bumper 2742 as a separate part 2744 from the flexible central portion 2710. An isometric view of the unassembled parts is shown in FIG. 27B. In this example, the separate part comprises a slot 2746 having the same width and thickness as the flexible central portion. The flexible central portion can slide through the slot. The flexible central portion also includes ridges 2712 designed to hold the separate part in place, with the ridges on either side of the slot.

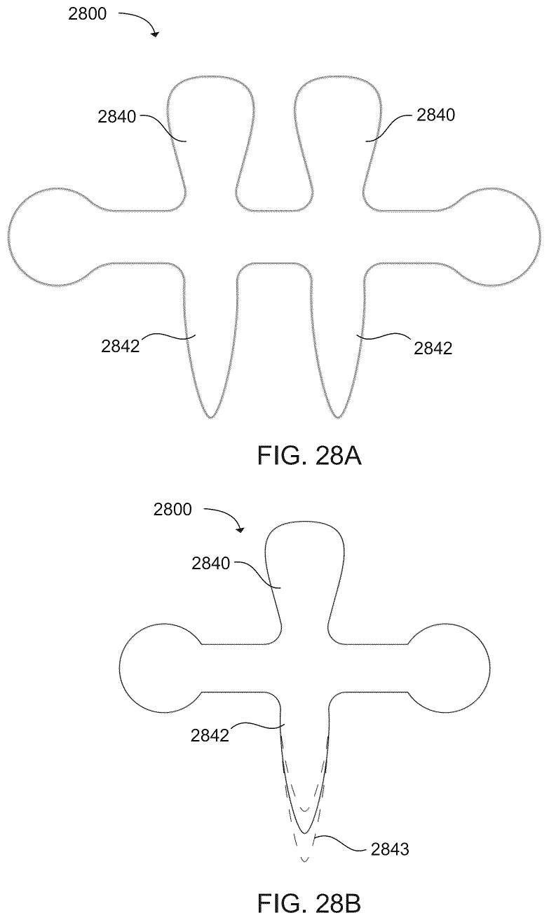

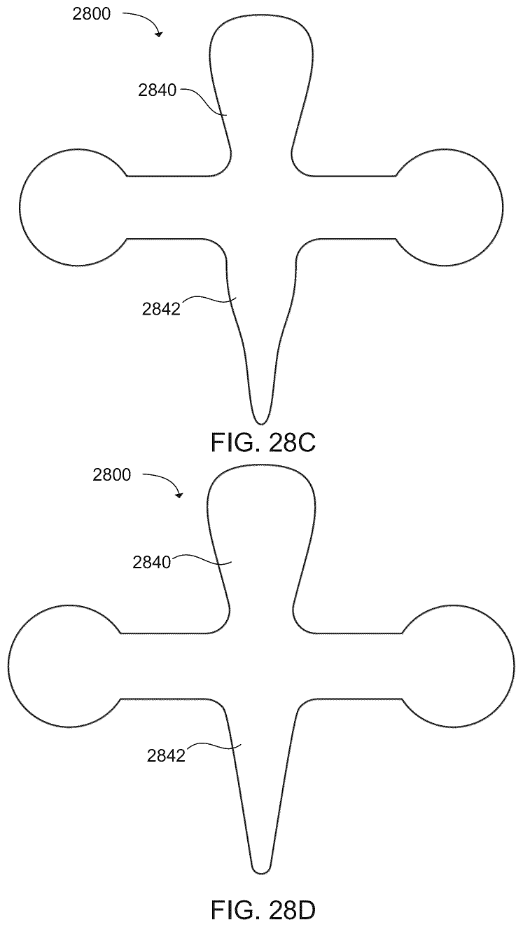

The example shown above have included a single dorsal bumper and a single volar bumper. However, in some examples, the flexible connector can have multiple dorsal bumpers, multiple volar bumpers, or both. FIG. 28A is a side view of an example flexible connector 2800 that includes multiple dorsal bumpers 2840 and multiple volar bumpers 2842. Additional options for the bumpers are shown in FIGS. 28B through and 28D. FIG. 28B shows a flexible connector with a volar bumper 2842 that has partially concave sides. FIG. 28C shows a flexible connector with a volar bumper that has straight sides leading to a tapered tip. FIG. 28D includes dashed outlines 2843 showing multiple different lengths of the volar bumper. The volar bumper can be designed to optimally facilitate joint bending. In particular, the volar bumper can be designed, as much as possible, to avoid creating an obstacle to bending when the joint the flexes.

Thus, the volar bumper can be a shape that does not block the motion of phalanges of the join when the joint is flexed. However, the volar bumper can still provide a cushion between the bones to prevent bone-on-bone contact. Overall, the volar bumper can be designed to provide a smooth force vs. position profile when flexing the joint. Ideally, the implantable device can allow for flexion with a low, constant force. Although this may not be completely achievable in all cases, the design of the volar bumper can help facilitate smooth flexing of the joint. The various shapes shown in FIGS. 28A-D can be used in some examples.

In further examples, the dorsal and volar bumpers may be made of different materials. For example, the dorsal and volar bumpers can be made of materials having a different hardness, a different elasticity, different strain-reactive properties, and so on. In certain examples, the dorsal bumper can be made from a strain rate-sensitive material, and the volar bumper can be made of a different material that is not strain rate-sensitive. In some examples, the dorsal and volar bumpers can be formed separately as separate parts using different materials, and then assembled together into a flexible connector. In other examples, the dorsal and volar bumpers can be formed from different materials, but co-formed as a single part. For example, two different materials can be injected into an injection mold to form the dorsal and volar bumpers from the two materials. Alternatively, the dorsal and volar bumpers can be made using additive manufacturing, and different materials can be used to form the dorsal and volar bumpers.

### Anchors for Implantable Devices for Arthroplasty

As mentioned above, the implantable devices for arthroplasty described herein can also include anchors. The anchors can be implanted into bones on one or both sides of a joint, and then the flexible connector can be inserted into the anchors to connect the anchors together and provide a hinge-like motion or non-linear arc of motion to the joint. In some examples, an anchor can include a hollow anchor body defining an interior cannula space. A slot can extend along a length of the anchor body. The slot can provide an opening from the interior cannula space to the exterior of the anchor body. The slot can have a slot width that is smaller than an internal width dimension in the interior cannula space. The anchor can also include a rounded edge at a transition between the slot and at least one of the interior surface of the anchor body or the exterior surface of the anchor body.

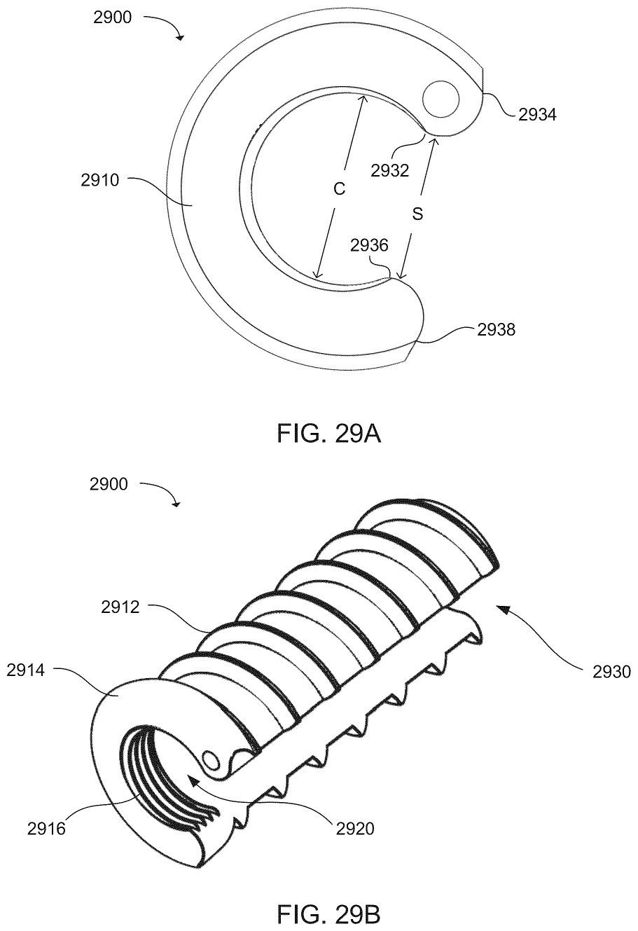

FIG. 29A shows an end view of one example anchor 2900. FIG. 29B shows an isometric view of the same example anchor. This anchor includes a hollow anchor body 2910 defining an interior cannula space 2920. A slot 2930 is formed along the length of the anchor body, and the slot provides an opening from the interior cannula space to the exterior of the anchor. The slot has a width(S) that is smaller than a width dimension of the cannula (C). As explained above, an anchoring portion of a flexible connector can be inserted into the cannula space, and the flexible central portion of the flexible connector can pass through the slot. However, because the slot has a smaller slot width than the width of the cannula, the anchoring portion can be trapped inside the cannula and prevented from pulling out of the anchor. The example shown in FIG. 29A also has rounded edges between the slot and the interior and exterior surfaces of the anchor body. The rounded edge include the upper interior edge 2932, the upper exterior edge 2934, the lower interior edge 2936, and the lower exterior edge 2938. The rounded edges can be useful to help prevent tearing or cutting of the flexible connector. If a slot were cut into an anchor without any rounded edges, the sharp edges of the slot may be prone to cut into the flexible material of the flexible connector, especially when the joint is repeatedly flexed. This example also includes threads 2912 on the exterior surface of the anchor body to allow the anchor to be screwed into a bone. A head portion 2914 is at the end of the anchor body. The head portion has a larger diameter than the rest of the anchor body, so that the anchor can be screwed into bone until the head portion abuts the bone surface. Additionally, the head portion includes internal threads 2916. These can be used to attach a threaded end cap (not shown).

For clarity, the longest dimension of the anchor is referred to as the “length” of the anchor. The length direction can also be referred to as the longitudinal axis of the anchor. In some examples, the anchor can be configured to be implanted transversely in a phalange, and the anchor is inserted into the phalange in the length direction. It should be noted that longitudinal axis of the anchor can be orthogonal to the longitudinal axis of the flexible connector. In the examples of the flexible connector described above, the dimension of the flexible connector referred as the width or lateral axis of the flexible connector can be parallel to the longitudinal axis of the anchor when the flexible connector is assembled with the anchor. With respect to the slot formed in the anchor body, the length of the slot can be in the same direction as the length of the anchor. The width of the slot can be orthogonal to the length of the slot, and the width can be the shortest distance across the slot. The upper side of the slot can refer to the side of the slot positioned higher when the anchor is implanted transversely, and the lower side of the slot can be the side that is positioned lower.

The anchor can have a rounded edge on at least one of the upper interior edge, the upper exterior edge, the lower interior edge, or the lower exterior edge of the slot. In some examples, all of these edges can be rounded. In other examples, only one edge can be rounded. In certain examples, the rounded edge can have a radius of curvature from about 0.05 mm to about 2 mm, or from about 0.1 mm to about 1.5 mm, or from about 0.1 mm to about 1 mm.

In certain examples, the upper interior edge and the lower interior edge of the slot can be rounded. The cannula can have a rounded shape such as a cylindrical shape. Together, the cannula and the rounded interior edges can form a teardrop-shaped profile. This example anchor can be used together with a flexible connector that has a matching teardrop-shaped profile of the anchoring portion of the flexible connector.

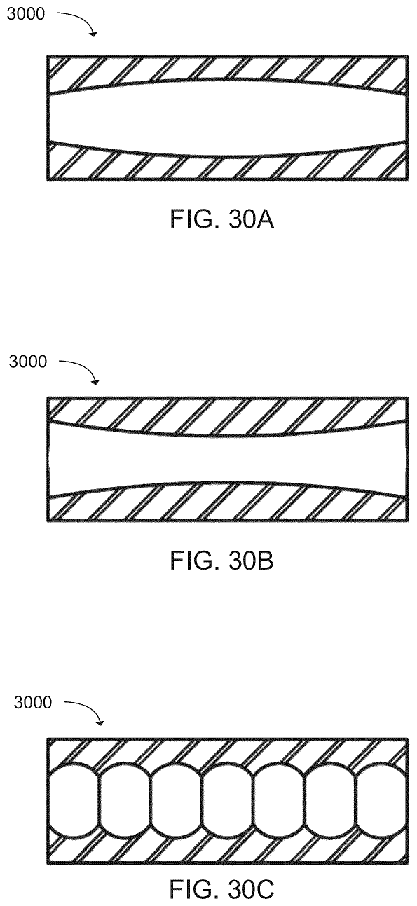

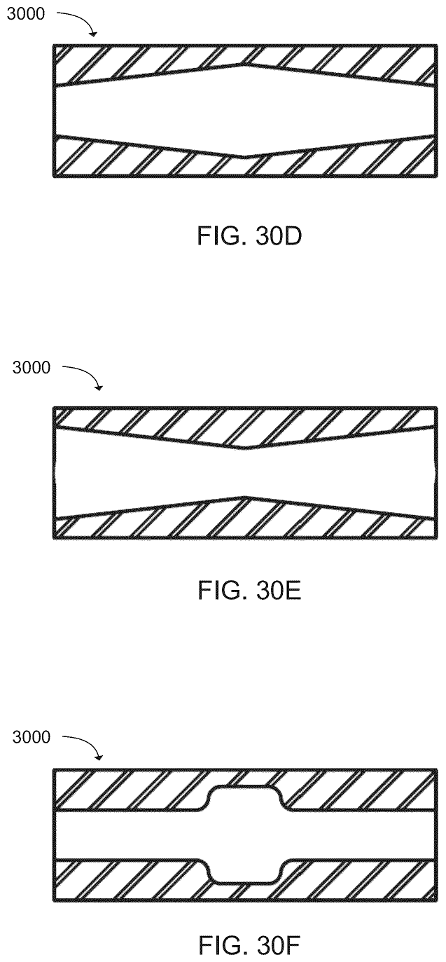

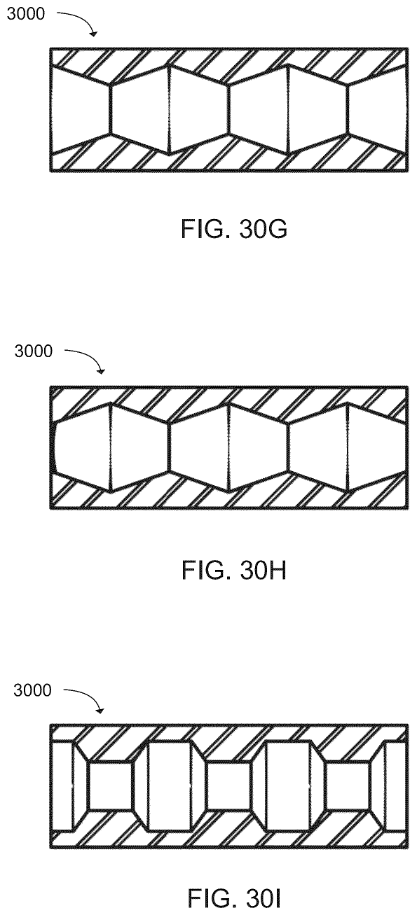

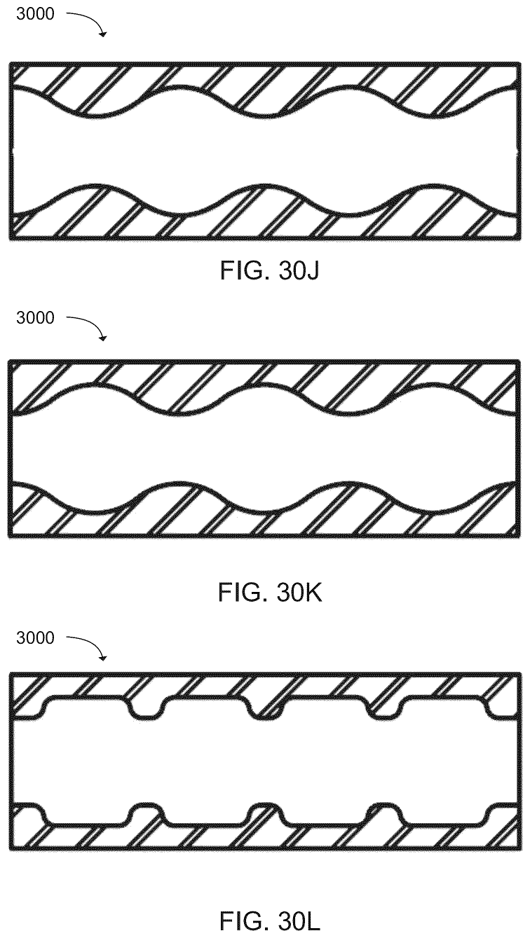

In some examples, the interior cannula space of the anchor can have a cylindrical shape, with a uniform diameter across the entire length of the cannula. However, in other examples, the cannula space can comprise a non-uniform internal diameter profile along a length of the anchor. This can facilitate retention of a flexible connector having an anchoring portion shaped to conform to the non-uniform diameter profile. The cannula space can be designed with any of the non-uniform diameter profiles described above for the anchoring portions of the flexible connectors. FIGS. 30A-30L show a variety of cross-sectional side views of anchors 3000 with a cannula space having various non-uniform diameter profiles. These have features matching the non-uniform thickness profiles of the flexible connectors shown in FIGS. 5A-5L. In some examples, the non-uniform internal diameter profile can have a narrowed portion at one end or both ends of the anchor and a larger-diameter portion between the ends. This type of anchor can be used with a flexible connector that has an anchoring portion tapered at both ends, and the tapered anchoring portion can be retained inside the anchor by the narrowed portions at the ends of the anchor.

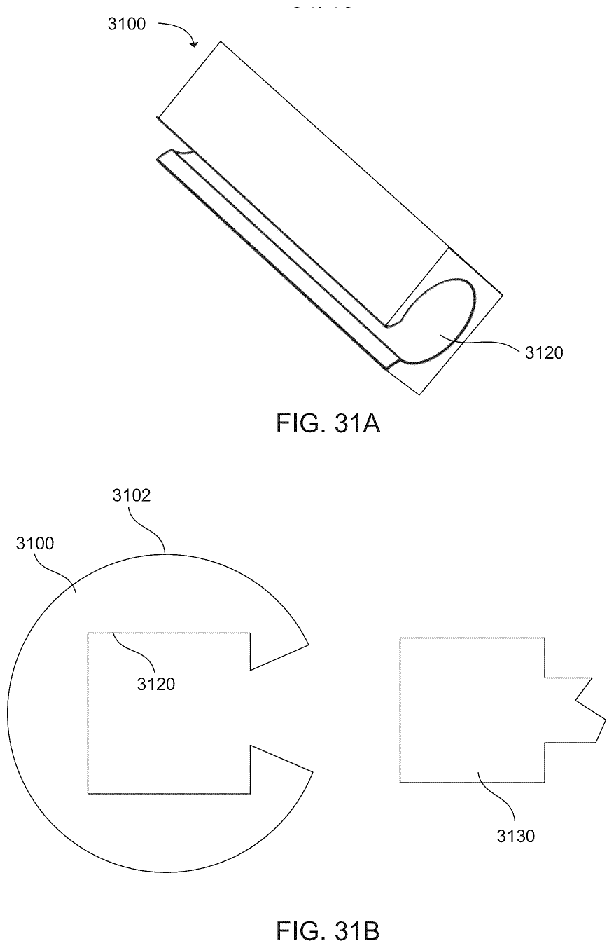

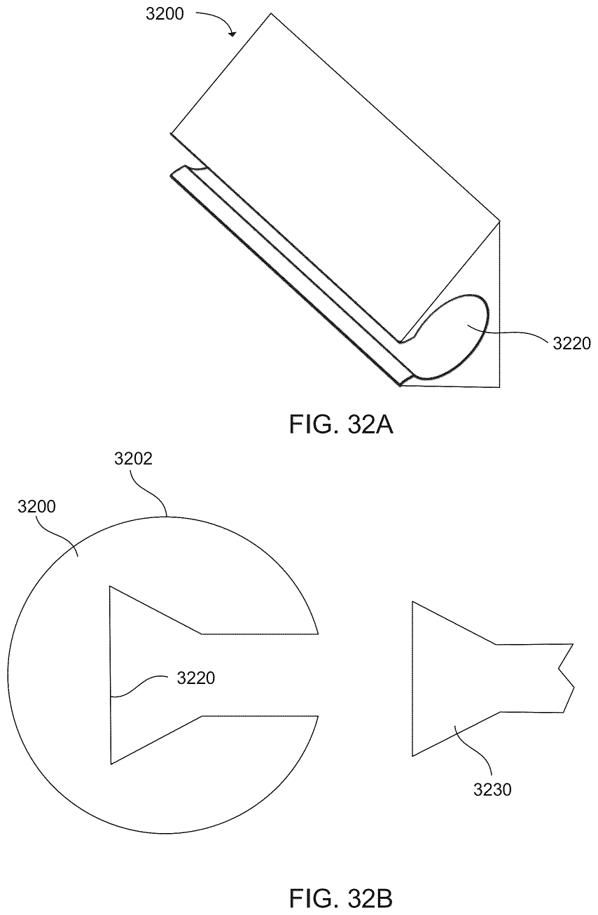

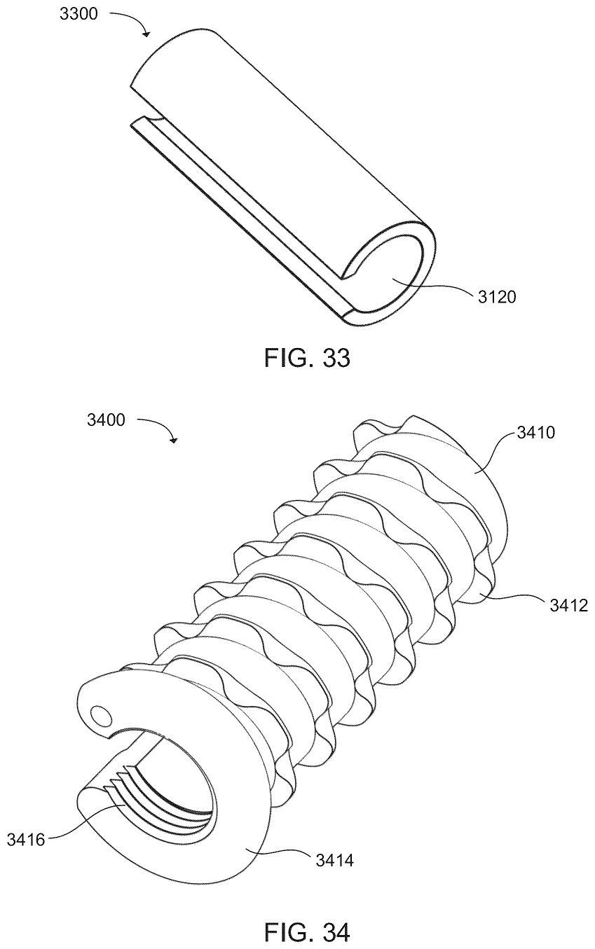

In some examples, the anchor can have a cylindrical shape, with a circular cross-section along the length of the anchor. In other examples, the anchor can have a differently shaped cross-section such as a square, triangular, hexagonal, oval, a dovetail groove shape, or other shape. FIG. 31A shows an example anchor 3100 with a square cross-sectional profile. However, the internal cannula 3120 of this anchor still has a cylindrical shape. In other examples, the internal cannula can have a different shape other than a circular cross-section. FIG. 31B is a side view of an example anchor 3100 that has an exterior surface 3102 with an outer cross-sectional profile that is circular, but an internal cannula 3120 cross-sectional profile that is square. This figure also shows an anchoring portion 3130 of a flexible connector that has a cross-sectional profile to match the internal cannula. This anchoring portion can fit into the internal cannula of the anchor. FIG. 32A shows an example anchor 3200 with a triangular cross-sectional profile on the exterior surface. This anchor also has an internal cannula 3220 with a cylindrical shape. FIG. 32B shows a different example anchor 3200 with an exterior surface 3202 that has a circular cross-sectional profile, and an internal cannula 3220 cross-sectional profile that is triangular. This figure also shows an anchoring portion 3230 of a flexible connector that matches the internal cannula cross-sectional profile. This anchoring portion can fit into the internal cannula having a matching shape. FIG. 33 shows another example anchor 3300 that has a circular cross-sectional profile. This anchor is shaped as a smooth cylinder on the outside and the anchor has an internal cannula 3320 with a cylindrical shape.

As mentioned above, in some examples the anchor body can have threads on an exterior surface to allow the anchor to be screwed into a bone. The threads can be discontinuous threads, because the threads do not extend across the slot in the side of the anchor. Additionally, the threads can have sharp points that can potentially cut into or tear the flexible material of the flexible connector. Therefore, in some examples the threads can have a depth that decreases gradually (i.e., the threads protrude from the surface less and less) approaching the sides of the slot. The threads can reach zero depth at or before the side of the slot (meaning that the threads do not protrude from the exterior surface of the anchor body immediately adjacent to the slot). This can help prevent the threads from cutting or tearing the flexible connector. The threads may also be discontinuous at other locations besides the slot. FIG. 34 shows an example anchor 3400 with discontinuous threads 3412 on the exterior surface of the anchor body 3410. This anchor also includes a head portion 3414 with internal threads 3416 for screwing in an end cap. In further examples, the threads can have a varying pitch along the length of the anchor. In certain examples, the anchor can also have a diameter that varies along the length of the anchor along with a thread pitch that varies along the length of the anchor.

The anchors described herein can be stemless, making them easier to implant transversely into phalanges. As an example, bores can be drilled transversely into a proximal phalange and a distal phalange on either side of a joint, and then anchors as described herein can be implanted into the bores. The anchors can have a smooth surface designed to be press-fit into the bores in some examples. In other examples, the anchors can have threads and the anchors can be screwed into the bores. In any of these examples, the anchors are inserted in a transverse direction. In certain examples, the anchors can have a length that is about equal to the whole width of the bone into the which the anchor is to be implanted. The anchor can then be implanted through the entire width of the bone.

The strength of the connection between the anchors and the bones in which they are implanted can be related to the geometry of the anchor and the surface area of the anchor that contacts the bone. Increasing the surface area can increase the strength of the connection between the anchor and the bone. In some examples, the anchor can include deep threads that provide more surface area in contact with bone. In some examples, the threads on the exterior surface of the anchor can be from about 1 mm to about 5 mm in depth, or from about 1 mm to about 3 mm, or from about 1 mm to about 2 mm, or from about 2 mm to about 5 mm, or from about 3 mm to about 5 mm in depth.

Although the examples described above have been stemless, the anchors described herein can incorporate stems in some examples to increase the strength of the connection between the anchor and the bone in which it is implanted. The stem can be a portion of the anchor extending from the exterior surface of the anchor body along the longitudinal axis of the phalange or other bone in which the anchor is implanted. The stem can extend orthogonally to the interior cannula of the anchor. The stem can extend in a direction away from the joint, opposite from the slot of the anchor, which faces toward the joint. This type of stemmed anchor can have a strong connection with the bone, but implanting this type of anchor can be more difficult and can require removing more bone.

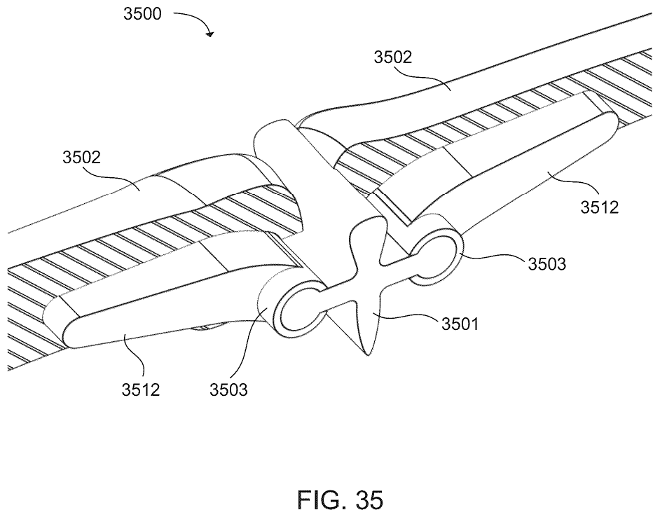

FIG. 35 shows a partial cutaway view of an example of a joint with an implantable device for arthroplasty 3500 implanted therein. The device includes two anchors 3503 that have stems 3512 extending away from the joint, along the longitudinal axis of the phalanges 3502. The anchors are connected by a flexible connector 3501 as described above.

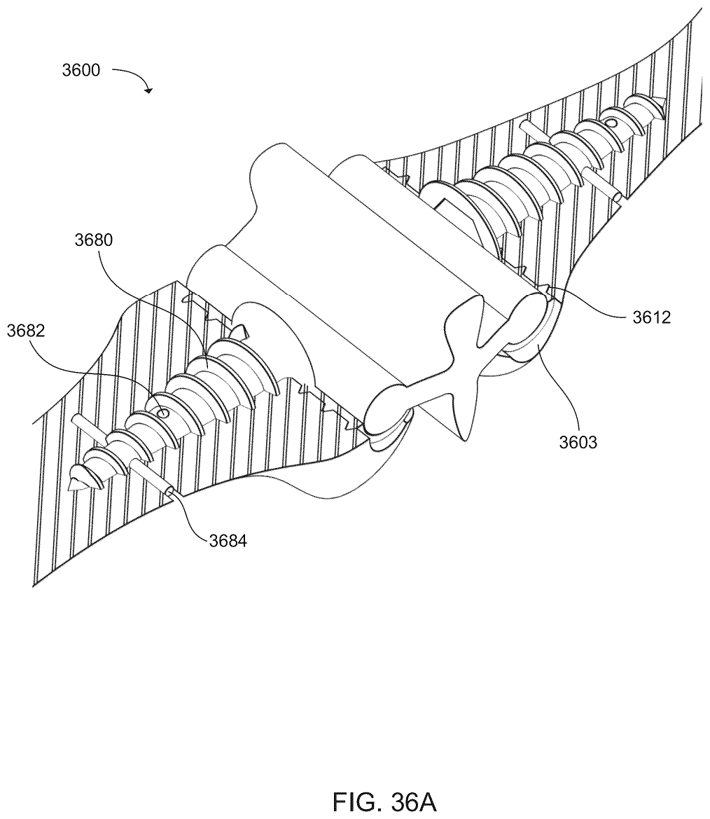

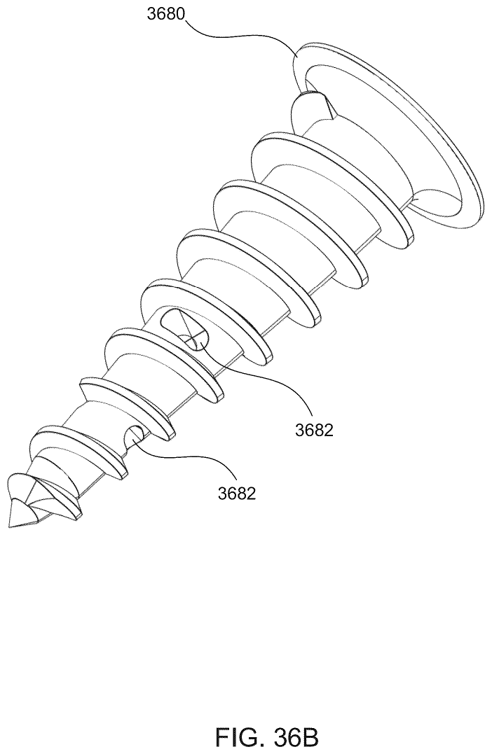

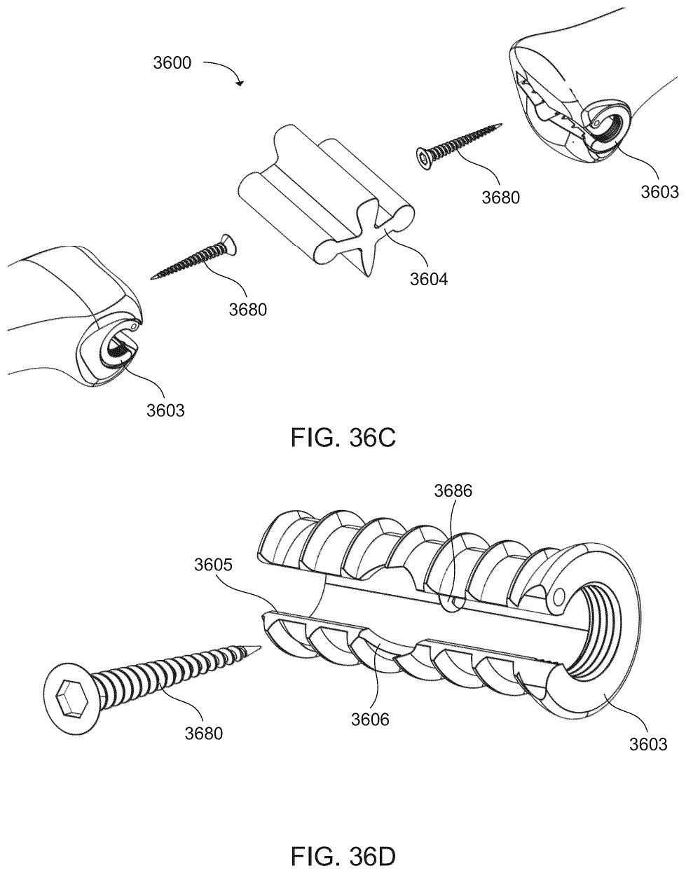

In further examples, the connection between the anchors and the bones can be strengthened by adding screws or other connectors to attach the anchors to the bone. FIG. 36 shows a cutaway view of a joint having another example implantable device for arthroplasty 3600 implanted therein. In this example, the anchors 3603 have threads 3612 to allow the anchors to be screwed into the bone. The anchors also include a screw hole leading from the interior cannula space out to the exterior surface of the anchor body, where the screw hole is positioned opposite from the slot. A screw 3680 is inserted through the screw hole and screwed into the phalange along the longitudinal axis of the phalange. This screw can strengthen the connection of the anchor to the bone. The screw also includes small holes 3682 for a transverse pin 3684 to be inserted through the screw to provide additional support. In this example, each screw includes two small holes oriented orthogonal one to another. This can ensure that at least one of the holes will be positions to allow a pin to be inserted transversely through the hole, no matter how the screw is rotationally oriented after being screwed into the bone. FIG. 36B shows a closer view of an example screw 3680. The screw has a wider head and narrows toward the tip. In this example, the holes 3682 are formed as slots that extend partially around the circumference of the screw. This can allow a transvers pin to be inserted through the hole from a wider variety of angles. Therefore, the screw can be driven into the bone and a pin can be inserted through one of the holes in the screw regardless of the rotational orientation of the screw. FIG. 36C shows an exploded view of the implantable device 3600, after the anchors 3670 have been implanted in the phalanges 3602 but before the screws 3680 have been driven into the phalanges through the screw holes in the anchors. A flexible connector 3604 is ready to be inserted into the anchors after the screws have been placed. FIG. 36D shows a closer view of an anchor 3603 and a screw 3680. The anchor includes a screw hole 3686. The screw can be driven through this screw hole into the bone in which the anchor is implanted. The anchor also includes a slot 3605 with cutouts 3606 that can allow the screw head to pass through when the screw is driven through the screw hole.

### Surgical Procedures for Implanting an Implantable Device for Arthroplasty

Surgical procedures can be used to implant any of the devices described above. As mentioned above, in some examples a flexible connector can be implanted directly into a joint without any separate anchors. In some examples, a surgical procedure can include: selecting a patient in need of implantation of an implantable device for arthroplasty; providing a flexible connector for an implantable device for arthroplasty; and implanting the flexible connector into the patient. The flexible connector can include any of the features and characteristics described herein. In more detail, the surgical procedure can include performing an incision at an implant site of the patient. The joint capsule and one or more phalangeal bones can then be prepared at the implant site. After preparing the bones, the flexible connector can be fitted into the implant site. A surgical wound closure can then be performed to close the implant site.

Preparing the phalangeal bones can involve forming a first cavity in a proximal phalange and forming a second cavity in a distal phalange. As used herein, the “proximal” phalange can be any phalange that is positioned closer to the patient's body, and the “distal” phalange can be any phalange that is position farther from the patient's body. It is to be understood that the phalanges are adjacent, i.e., sharing a joint between the phalanges. The cavities can be formed near the ends of the phalanges that face each other in the joint. In certain examples, the cavities can be formed by drilling bores transversely into the phalanges. The bores can extend part way through the bone or completely through the bone. In certain examples, the bores can be drilled into the cortical bone on one side of the phalange, through the cancellous bone, but the bore can stop before reaching the cortical bone on the opposite side of the phalange. However, in other examples, the bore can be drilled through the cortical bone on both sides of the phalange.

Preparing the phalangeal bones can also involve forming a slot. A first slot can be formed in the proximal phalange and a second slot can be formed in the distal phalange. The first slot and the second slot can face one another, and the slots can form an open pathway between the first cavity and the second cavity. In particular, the slots can face one another and form a straight pathway between the cavities when the joint is in an extended position. when the joint is in a flexed position, the slots can be oriented at an angle one to another.

After preparing the cavities and slots in the phalangeal bones, a flexible connector can be fitted into the implant site. A first anchoring portion of the flexible connector can fit into the first cavity and a second anchoring portion of the flexible connector can fit into the second cavity. The flexible central portion of the flexible connector can extend through the slots to connect the anchoring portions together. If the flexible connector has a dorsal bumper or a volar bumper, the bumper can be positioned between the proximal phalange and the distal phalange. As mentioned above, fitting the flexible connector into the implant site can be done without any separate anchor pieces in some examples. In a certain example, the flexible connector can be press-fit into the cavities in the phalanges, and the flexible connector can be held in place at least partially by friction.

In further examples, the flexible connector can include rod sleeves configured to accept fixation rods. The surgical procedure can include inserting the fixation rods into the rod sleeves. In some examples, the flexible connector can be inserted into the cavities in the phalanges first, and then the fixation rods can be inserted. The fixation rods can expand the anchoring portions of the flexible connector to increase friction between the anchoring portions and the bone surrounding the anchoring portions, thus facilitating retention of the anchoring portions in the cavities.

Alternatively, surgical procedures can be used to implant an implantable device for arthroplasty that includes at least one anchor and a flexible connector. In some examples, the device can include two anchors, where one anchor is implanted into each phalange of the joint. The flexible connector can then be fitted into the anchors. The two anchors can be identical in some examples, while in other examples the two anchors can be different. In certain examples, one anchor can be a mirror of the other, i.e., features of the anchor can be inverted as in a mirror image. In other examples, the anchors may be designed or sized differently to accommodate different sizes or shapes of the phalanges into which the anchors are to be implanted.

In some examples, a surgical procedure for implanting an implantable device for arthroplasty can include: selecting a patient in need of implantation of an implantable device for arthroplasty; providing two anchors; providing a flexible connector configured to be held between the two anchors; and implanting the anchors and the flexible connector into the patient. In certain examples, the procedure can also include performing an incision at an implant site of the patient. The joint capsule can then be prepared and two phalangeal bones can be prepared at the implant site. The anchors can then be fitted into the two phalangeal bones. The flexible connector can be fitted into the anchors. A surgical wound closure can then be performed to close the implant site.

In some examples, preparing the phalangeal bones can include forming a first cavity in a proximal phalange and forming a second cavity in a distal phalange. One of the anchors can fit transversely into the first cavity. This anchor can be referred as the proximal anchor. The other anchor can fit transversely into the second cavity. This anchor can be referred to as the distal anchor. The anchors can be positioned so that the slots of the anchors are aligned and face toward one another when the joint is in an extended position. The surgical method can also include forming a first slot in the proximal phalange and forming a second slot in the distal phalange. The first slot formed in the phalange can be aligned with the slot of the proximal anchor. Similarly, the second slot in the distal phalange can be aligned with the slot of the distal anchor. The slots in the phalanges can be cut using a tool such as a bone saw. In various examples, the slots in the phalanges can be cut either before or after the anchors are implanted. In certain examples, it can be useful to implant the anchors first and then cut the slots in the bone after the anchors are in place, because the bones can be stronger before the slots are cut. This can reduce the risk of break a portion of the bone when implanting the anchors.

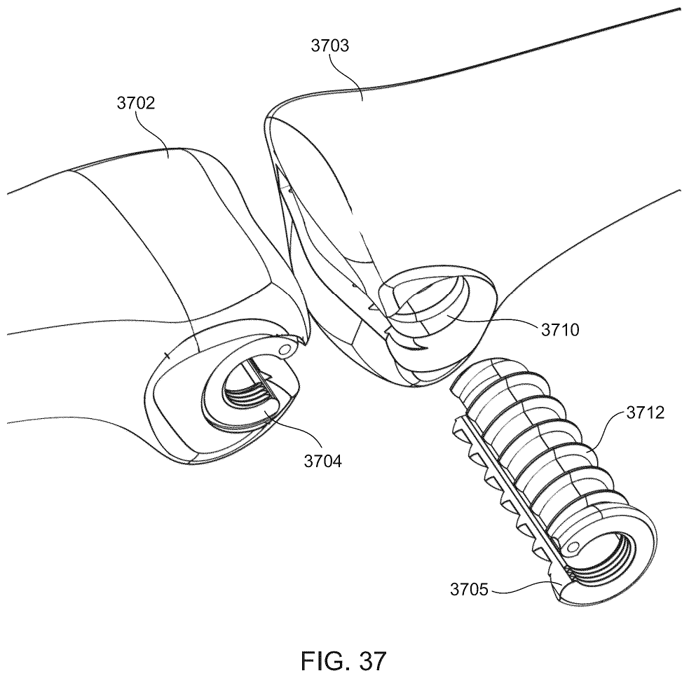

FIG. 37 shows an example in which a proximal anchor 3704 has been implanted in a proximal phalange 3702, and a distal anchor 3705 is prepared to be implanted into a distal phalange 3703. In this example, a bore 3710 has been drilled in each phalange. The bore has a diameter sized to accept the anchors. The anchors in this example include threads 3712, which allow the anchors to be screwed into the bores. The threads can extend outward to engage with the bone surrounding the bores.

As mentioned above, the anchors can be implanted so that the slots of the anchors face one another when the joint is in an extended position. The anchors can also be inserted to a particular desired insertion depth, which may depend on the width of the phalange, the length of the anchor, and other factors. If the anchors have exterior threads for screwing the anchors into the bone, then the rotational orientation of the anchor is constantly changing as the anchor screws into the bone. It can be useful to stop screwing the anchor into the bone when the slot is oriented in the appropriate direction to line up with the slot of the opposite anchor. In some examples, the surgical procedure can include determining an appropriate initial rotational position of the anchor before screwing the anchor into the bone, so that when the anchor reaches the desired insertion depth it will be in the correct rotational position for the slot to align with the slot of the other anchor. The distance that the anchor will screw in with each full rotation can be related to the pitch of the threads. Therefore, the appropriate starting rotational position for the anchor can be pre-calculated using the pitch of the threads and the desired insertion depth. In some examples, the surgical procedure can include pre-calculating this starting rotational position before beginning to screw in the anchor. This can be done for one anchor or for both anchors.

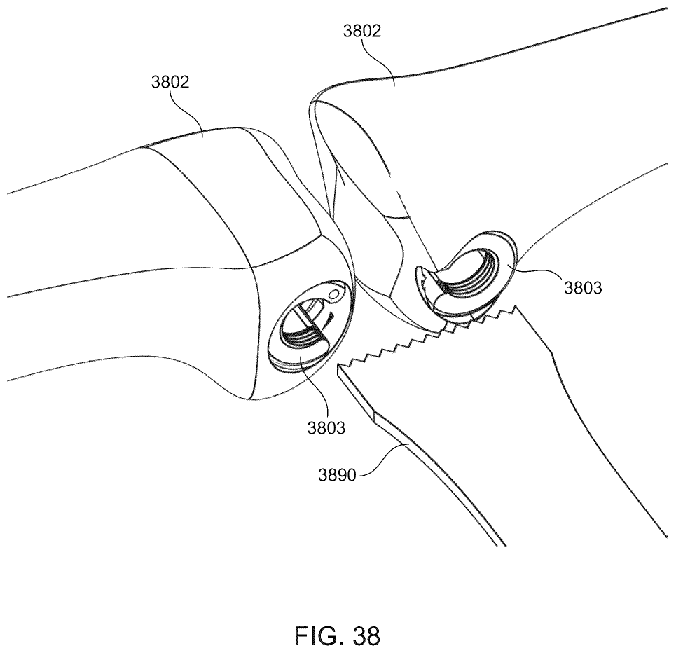

FIG. 38 shows an example where two anchors 3803 have been screwed into phalanges 3802. The anchors were screwed in to an insertion depth at which their slots are aligned and facing one toward another while the joint is in the extended position. In this example, the anchors were implanted before cutting slots in the phalanges. This figure shows a bone saw 3890 that can be used to cut slots in the phalanges. The slots of the anchors can be used as a guide for cutting the slots in the phalanges, so that all the slots are aligned one with another. After forming the slots in the phalanges, a flexible connector can be implanted.

The surgical procedures described herein can also make use of surgical guides, such as pins, wires, jogs, and so on. In some examples, guide pins, guide wires, and/or a jig can be used to drill parallel bores in the phalanges. This can allow accurate alignment of the anchors. The surgical guides can also be configured to locate the bores at an appropriate location in the phalange bone. When cutting slots, surgical guides can also be used. For example, a cutting jig and/or alignment guide can be used when cutting the slots to ensure the slots are cut accurately and that the slots align one with another.

In further examples, it may be useful to adjust the alignment of the implantable device after the anchors have been implanted and the slots have been formed. For example, in some cases the anchors may be slightly non-aligned after being implanted. In some examples, the alignment can be adjusted by using a flexible connector that has anchoring portions that are non-aligned in a way that matches the anchors. Flexible connectors can be manufactured with multiple slightly different alignments between the anchoring portions so that a flexible connector can be chosen to fit with non-aligned anchors. Alternatively, shims can be inserted between the anchoring portion of the flexible connector and the anchor to adjust the alignment of the flexible connector with respect to the anchor. In other examples, the flexible connector can be made of a material that is re-moldable or re-shapeable after implanting. The shape of the flexible connector can then be changed slightly after implanting to account for non-alignment of anchors.

As explained above, the anchors and flexible connectors described herein can be configured to fit together. In particular, the anchoring portions of the flexible connector can be configured to fit into and be retained by the internal cannula space of the anchor. The flexible central portion of the flexible connector can be configured to pass through the slot of the anchor. In some cases, multiple different flexible connectors can be designed that can fit together with a particular anchor. For example, different flexible connectors can be designed with different dorsal bumpers or volar bumpers, or different shapes of the flexible central portion, or different lengths, or different materials that have different elasticity or other properties. If these different flexible connectors are designed with similar anchoring portions, then the different flexible connectors can still be used with the same anchors.

In some cases, it may be useful to replace a flexible connector that has already been implanted with another flexible connector. The new flexible connector can have a different design from the already-implanted flexible connector, as long as the new flexible connector has anchoring portions configured to fit into the anchors that are already implanted. In some cases, it can be useful to replace a flexible connector with a different flexible connector having a different length or width, or differently shaped bumpers, or a different elasticity, or other different properties. Alternatively, the new flexible connector can have the same design as the already-implanted flexible connector. This can be useful if the already-implanted flexible connector shows signs of wear or degradation, such as tearing or other defects. A surgical procedure can be performed to replace the flexible connector without disturbing the anchors that are already implanted. Thus, the flexible connector can be replaced without significantly impacting the bones of the joint.

In one example, a surgical procedure for replacing a flexible connector of an implanted device for arthroplasty can include identifying a patient having an already-implanted device for arthroplasty that includes two anchors and a flexible connector held between the two anchors. An incision can be performed at the implant site of the patient. The flexible connector can then be removed from the anchors. A replacement flexible connector can be fitted into the anchors, and the replacement flexible connector can include anchoring portions that are compatible with the anchors. A surgical would closure can then be performed to close the implant site.

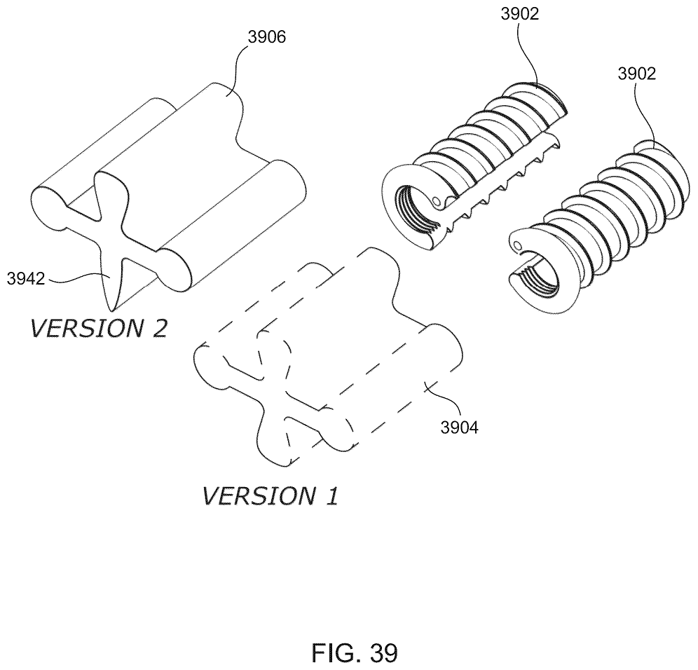

FIG. 39 shows an example of a replacement flexible connector 3906 that can be used to replace a previously-implanted flexible connector 3904. In this example, the replacement flexible connector has a differently shaped volar bumper 3942 than the previously-implanted flexible connector. The anchors 3902 can be already-implanted in a joint (not shown). Both the replacement flexible connector and the previously-implanted flexible connector can include anchoring portions that are compatible with the anchors.

The combination of anchors and flexible connector, along with any other components such as fixation rods, end caps, additional screws and pins, or other components, can be referred to as an implantable device for arthroplasty. Thus, the surgical methods described herein can include implanting any of these components.

In certain examples, an implantable device for arthroplasty can include a first anchor, a second anchor, and a flexible connector. The anchors and the flexible connector can include any of the features and characteristics described above. The anchors and flexible connector can also be sized and shaped to match one another. For example, the anchors can have interior cannula spaces that are sized and shaped to match the size and shape of the anchoring portions of the flexible connector. In further examples, the anchors can be designed to have smooth, curved surfaces in locations that contact the flexible connector. For example, the anchors can be designed so that exterior threads do not contact the flexible connector to prevent the threads from cutting or tearing the flexible connector. The anchors can also have slots with one or more rounded edges to prevent the edges from cutting or tearing the flexible connector. In certain examples, the anchors can have a rounded interior cannula and rounded interior edges of the slot, where the interior cannula and the rounded interior edges together form a teardrop-shaped profile. The flexible connector can have an anchoring portion that has a matching teardrop-shaped profile that fits into the anchor. In further examples, the flexible connector can be designed with a greater width in a central portion of the flexible connector than at the anchoring portions. The anchoring portions can have a first width that fits within the anchors, but the central portion of the flexible connector can have a greater width. In certain examples, the greater width can be substantially the full width of a joint capsule in which the device is to be implanted.

It is to be understood that the examples of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular examples only and is not intended to be limiting.

As used herein, a plurality of items, structural elements, compositional elements, and/or materials can be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various examples of the present invention can be referred to herein along with alternatives for the various components thereof. It is understood that such examples and alternatives are not to be construed as de facto equivalents of one another, but are to be considered as separate and autonomous representations of the present technology.

Furthermore, the described features, structures, or characteristics can be combined in any suitable manner in one or more examples. In the description, numerous specific details are provided, such as examples of lengths, widths, shapes, etc., to provide a thorough understanding of examples of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description. Reference throughout this specification to “one example” or “an example” means that a particular feature, structure, or characteristic described in connection with the example is included in at least one example of the present invention. Thus, appearances of the phrases “in one example” or “in an example” in various places throughout this specification are not necessarily all referring to the same example.

Although the disclosure may not expressly disclose that some examples or features described herein may be combined or interchanged with other examples or features described herein, this disclosure should be read to describe any such combinations that would be practicable by one of ordinary skill in the art no matter the specific examples that were described. Indeed, unless a certain combination of elements or functions not expressly disclosed would conflict with one another, such that the combination would render the resulting example inoperable or impracticable as would be apparent to those skilled in the art, this disclosure is meant to contemplate that any disclosed element or feature or function in any example described herein can be incorporated into any other example described herein (e.g., the elements or features or functions combined or interchanged with other elements or features or functions across examples) even though such combinations or interchange of elements or features or functions and resulting examples may not have been specifically or expressly disclosed and described. Indeed, the following examples are further illustrative of several embodiments of the present technology:
1. A flexible connector for an implantable device for arthroplasty, comprising:
   a flexible central portion;
   a first anchoring portion extending from a first end of the central portion; and
   a second anchoring portion extending from a second end of the central portion, opposite from the first end;
   wherein the first anchoring portion and the second anchoring portion each comprise a thickened portion located farther away from the central portion and a thinner portion, relative to the thickened portion, located nearer to the central portion than the thickened portion.
2. The flexible connector of example 1, wherein the flexible central portion, first anchoring portion, and second anchoring portion are integrally formed from a flexible material as a single part.
3. The flexible connector of example 2, wherein the flexible material comprises silicone, medical grade plastic, biocompatible elastic polymer, polyetheretherketone (PEEK), polypropylene (PE), ultra-high molecular weight polyethylene (UHMWPE), polypropylene (PP), polytetrafluoroethylene (PTFE), perfluoroalkoxy (PFA), fluorinated ethylene propylene (FEP), polyurethane (PU), thermoplastic polyurethane (TPU), or a combination thereof.
4. The flexible connector of any of examples 1-3, wherein at least one of the first anchoring portion or the second anchoring portion is formed as a separate part from the flexible central portion.
5. The flexible connector of any of examples 1-4, wherein the first anchoring portion and the second anchoring portion comprise a transition portion extending from the thickened portion to the flexible central portion, wherein the transition portion is devoid of concave edges and wherein the transition portion does not join the flexible central portion at a concave edge.
6. The flexible connector of example 5, wherein the transition portion comprises a concave curved surface having a radius of curvature from about 0.1 mm to about 10 mm.
7. The flexible connector of any of examples 1-6, wherein at least one of the first anchoring portion or the second anchoring portion has a teardrop-shaped profile transitioning smoothly to the flexible central portion.
8. The flexible connector of any of examples 1-7, wherein at least one of the first anchoring portion or the second anchoring portion comprises a lateral taper to facilitate lateral insertion.
9. The flexible connector of any of examples 1-8, wherein at least one of the first anchoring portion or the second anchoring portion comprises a non-uniform thickness profile to facilitate retention in an anchor having a cavity shaped to conform to the non-uniform thickness profile.
10. The flexible connector of any of examples 1-9, wherein at least one of the first anchoring portion or the second anchoring portion comprises a rod sleeve configured to accommodate a fixation rod, the rod sleeve extending along a width direction of the flexible connector.
11. The flexible connector of example 10, further comprising a fixation rod configured to be held in the rod sleeve.
12. The flexible connector of example 11, wherein the rod is integrally formed with the fixation rod inside the rod sleeve.
13. The flexible connector of any of examples 11-12, wherein the fixation rod is a separate piece configured to be inserted into the rod sleeve after implantation of the flexible connector.
14. The flexible connector of any of examples 11-13, wherein the fixation rod has a surface feature comprising threads, one or more annular ribs, barbs, a smooth surface, or a combination thereof.
15. The flexible connector of any of examples 11-14, wherein the fixation rod has a non-circular cross section.
16. The flexible connector of any of examples 11-15, wherein the fixation rod comprises a non-uniform thickness profile to facilitate retention in the rod sleeve, wherein the rod sleeve comprises a cavity shaped to conform to the non-uniform thickness profile.
17. The flexible connector of any of examples 11-16, wherein the rod sleeve comprises an entrance portion having a sleeve diameter less than a diameter of the fixation rod and a retention portion having a diameter substantially matching the diameter of the fixation rod.
18. The flexible connector of any of examples 11-17, wherein the fixation rod comprises an insertion portion configured to be inserted into the rod sleeve and an end cap portion at an end of the fixation rod, the end cap portion having a diameter greater than a diameter of the insertion portion.
19. The flexible connector of any of examples 11-18, wherein the fixation rod has a length sufficient to extend across an entire width of the first anchoring portion or the second anchoring portion.
20. The flexible connector of any of examples 11-19, wherein the fixation rod has a length less than a width of the first anchoring portion or the second anchoring portion.
21. The flexible connector of any of examples 1-20, wherein the width of the flexible central portion increases from a first width adjacent to at least one of the first anchoring portion or the second anchoring portion, to a greater second width between the first anchoring portion and the second anchoring portion.
22. The flexible connector of example 21, wherein the second width is substantially a full width of a joint capsule into which the flexible connector is to be implanted.
23. The flexible connector of any of examples 1-22, wherein the flexible central portion comprises a wave-shaped portion to facilitate extension of the flexible central portion along a longitudinal axis.
24. The flexible connector of any of examples 1-23, further comprising a plurality of fibers extending along a longitudinal axis of the flexible connector, the fibers configured to define a maximum extension of the flexible connector.
25. The flexible connector of example 24, wherein the fibers are embedded within the flexible central portion.
26. The flexible connector of example 25, wherein the fibers have a length that is longer than a relaxed length of the flexible connector.
27. The flexible connector of example 24, wherein the fibers are adhered to a surface of the flexible central portion.
28. The flexible connector of any of examples 24-27, wherein the fibers are attached to or wrapped around at least one of a first fixation rod within the first anchoring portion or a second fixation rod with the second anchoring portion.
29. The flexible connector of any of examples 1-28, further comprising a dorsal bumper extending from a dorsal surface of the flexible central portion and a volar bumper extending from a volar surface of the flexible central portion.
30. The flexible connector of example 29, wherein the dorsal bumper has an upper bulged portion to prevent contact between a proximal phalange and a distal phalange.
31. The flexible connector of example 30, wherein the upper bulged portion is shaped to conform to a surface of the proximal phalange and a surface of the distal phalange.
32. The flexible connector of any of examples 30-31, wherein the upper bulged portion comprises a strain rate-sensitive material configured to conform to a surface of the proximal phalange and a surface of the distal phalange.
33. The flexible connector of any of examples 29-32, wherein the volar bumper tapers to a lower narrowed portion to facilitate flexion of a joint into which the flexible connector is to be implanted.
34. The flexible connector of any of examples 29-33, wherein the dorsal bumper and the volar bumper are integrally formed with the flexible central portion as a single part.
35. The flexible connector of example 34, wherein the dorsal bumper and the volar bumper are devoid of concave edges and wherein the dorsal bumper and the volar bumper do not join the flexible central portion at a concave edge.
36. The flexible connector of example 35, wherein the dorsal bumper and the volar bumper comprise a concave curved surface joining the flexible central portion, the concave curved surface having a radius of curvature from about 0.1 mm to about 10 mm.
37. The flexible connector of any of examples 34-36, wherein the flexible central portion comprises a varying durometer hardness over a length of the flexible central portion, wherein the varying durometer hardness comprises a first stiffness adjacent to at least one of the dorsal bumper or the volar bumper and a second stiffness farther from the dorsal bumper or the volar bumper, wherein the first stiffness is greater than the second stiffness.
38. The flexible connector of any of examples 34-37, wherein the flexible central portion has a varying thickness over a length of the flexible central portion, wherein the thickness adjacent to at least one of the dorsal bumper or the volar bumper is greater than thickness farther from the dorsal bumper or the volar bumper.
39. The flexible connector of any of examples 29-38, wherein the dorsal bumper and the volar bumper are formed as a separate part from the flexible central portion, configured to be attached to the flexible central portion.
40. The flexible connector of example 39, wherein the separate part comprises an opening configured to allow the connector body to extend through the opening.
41. The flexible connector of example 40, wherein at least one of the separate part or the flexible central portion comprises a surface alignment feature to facilitate alignment of the separate part with the flexible central portion.
42. The flexible connector of any of examples 29-41, wherein the flexible connector comprises multiple dorsal bumpers, multiple volar bumpers, or both.
43. A surgical procedure for implanting an implantable device for arthroplasty, comprising:
   selecting a patient in need of implantation of an implantable device for arthroplasty;
   providing a flexible connector for an implantable device for arthroplasty as in any of examples 1-42; and
   implanting the flexible connector into the patient.
44. The surgical procedure of example 43, further comprising:
performing an incision at an implant site of the patient;
   preparing a joint capsule and one or more phalangeal bones at the implant site;
   fitting the flexible connector into the implant site; and
   performing a surgical closure of a wound at the implant site.
45. The surgical procedure of example 44, wherein preparing the one or more phalangeal bones comprises:
forming a first cavity in a proximal phalange optionally using a surgical guide;
forming a second cavity in a distal phalange optionally using a surgical guide;
forming a first slot in the proximal phalange and a second slot in the distal phalange, optionally using a cutting guide, wherein the first slot and the second slot face one another; and
wherein fitting the flexible connector into the implant site comprises fitting the first anchoring portion into the first cavity and fitting the second anchoring portion into the second cavity such that the flexible central portion body extends through the first slot and the second slot.
46. The surgical procedure of example 45, wherein at least one of the first anchoring portion or the second anchoring portion is press-fit into the respective cavity without a separate anchor part.
47. The surgical procedure of any of examples 45-46, further comprising inserting a fixation rod into a rod sleeve in at least one of the first anchoring portion or the second anchoring portion.
48. A flexible connector for an implantable device for arthroplasty, comprising: a flexible bridge member having a first segment extending from a reference axis in a first direction and terminating at a first end and a second segment extending from the reference axis in a second direction and terminating at a second end, the flexible bridge member being configured to flex, rotate, translate, or a combination thereof, about the reference axis;
   a first anchor interface located at the first end; and
   a second anchor interface located at the second end;
   wherein the first anchor interface comprises a thickness greater than a thickness of the first segment of the flexible bridge member; and
   wherein the second anchor interface comprises a thickness greater than a thickness of the second segment of the flexible bridge member.
49. An anchor for an implantable device for arthroplasty, comprising:
   a hollow anchor body defining an interior canula space;
   a slot extending along a length of the anchor body, the slot providing an opening from the interior cannula space to an exterior of the anchor body, wherein the slot has a slot width that is smaller than an internal width dimension in the interior cannula space; and
   a rounded edge at a transition between the slot and at least one of an interior surface of the anchor body or an exterior surface of the anchor body.
50. The anchor of example 49, wherein the rounded edge is only present along one side of the slot.
51. The anchor of any of examples 49-50, wherein the rounded edge is present along both sides of the slot.
52. The anchor of any of examples 49-51, wherein the rounded edge comprises rounded edges present at the transitions from the slot to the interior surface along both sides of the slot, and wherein the rounded edges and the interior cannula space together have a teardrop-shaped profile.
53. The anchor of any of examples 49-52, wherein the interior cannula space comprises a non-uniform internal diameter profile along a length of the anchor to facilitate retention of a flexible connector having an anchoring portion shaped to conform to the non-uniform internal diameter profile.
54. The anchor of example 53, wherein the non-uniform internal diameter profile comprises a narrowed portion at one end or both ends of the anchor and a larger-diameter portion between the ends.
55. The anchor of any of examples 49-54, wherein the exterior surface of the anchor body has a smooth, cylindrical shape.
56. The anchor of any of examples 49-55, wherein the exterior surface of the anchor body has a cross-sectional profile selected from circular, square, triangular, hexagonal, oval, or a dovetail groove shape.
57. The anchor of any of examples 49-56, wherein the exterior surface of the anchor body comprises threads configured to allow the anchor body to be screwed into a phalangeal bone.
58. The anchor of any of examples 49-57, wherein the threads are discontinuous at the slot.
59. The anchor of example 58, wherein a depth of the threads decreases gradually toward the slot such that the threads do not protrude from the exterior surface of the anchor body adjacent to the slot.
60. The anchor of any of examples 58-59, wherein the threads comprise at least one other discontinuity at another location besides the slot.
61. The anchor of any of examples 49-60, wherein the anchor body is configured to be implanted transversely through an entire width of a phalangeal bone.
62. The anchor of any of examples 49-61, further comprising a stem extending from the exterior surface of the anchor body, opposite from the slot and orthogonal to the interior cannula space.
63. The anchor of any of examples 49-62, further comprising a screw hole in the anchor body, the screw hole extending from the interior surface to the exterior surface, the screw hole being positioned opposite from the slot.
64. The anchor of example 63, further comprising a fixation screw configured to be screwed through the screw hole into a phalangeal bone along the length of the phalangeal bone.
65. The anchor of example 64, wherein the fixation screw increases in diameter toward the anchor when the screw is screwed in place.
66. The anchor of any of examples 64-65, wherein the fixation screw comprises a hole extending transversely through a shaft of the screw to allow a pin to be inserted through the hole after the screw has been screwed in place.
67. The anchor of any of examples 64-66, wherein the fixation screw comprises two holes extending through a shaft of the screw, wherein the two holes are orthogonal one to another, to allow a pin to be inserted transversely through one of the holes after the screw has been screwed in place.
68. A surgical procedure for implanting an implantable device for arthroplasty, comprising:
   selecting a patient in need of implantation of an implantable device for arthroplasty;
   providing two anchors as in any of examples 49-67;
   providing a flexible connector configured to be held between the two anchors; and
   implanting the anchors and the flexible connector into the patient.
69. The surgical procedure of example 68, further comprising:
   performing an incision at an implant site of the patient;
   preparing a joint capsule and two phalangeal bones at the implant site;
   fitting the anchors into the two phalangeal bones;
   fitting the flexible connector into the anchors; and
   performing a surgical closure of a wound at the implant site.
70. The surgical procedure of example 69, wherein preparing the two phalangeal bones comprises:
   forming a first cavity in a proximal phalange optionally using a surgical guide;
   forming a second cavity in a distal phalange optionally using a surgical guide;
   forming a first slot in the proximal phalange and a second slot in the distal phalange, optionally using a cutting guide, wherein the first slot and the second slot face one another;
   wherein a proximal anchor of the two anchors is fit transversely into the first cavity and a distal anchor of the two anchors is fit transversely into the second cavity such that slots of the anchors are aligned with the first slot and the second slot.
71. The surgical procedure of example 70, wherein the flexible connector comprises a first anchoring portion held in the proximal anchor, a second anchoring portion held in the distal anchor, and a flexible central portion extending from the first anchoring portion, through the slots of the anchors and the proximal and second slots of the phalangeal bones, to the second anchoring portion.
72. The surgical procedure of any of examples 70-71, wherein the proximal anchor is press-fit into the first cavity, or wherein the distal anchor is press-fit into the second cavity, or both.
73. The surgical procedure of any of examples 70-72, wherein at least one of the proximal anchor or the distal anchor comprises threads on an exterior surface, and wherein at least one of the proximal anchor or the distal anchor is fit by screwing into bone.
74. The surgical procedure of example 73, further comprising pre-calculating a starting rotational position for at least one of the proximal anchor or the distal anchor based on a pitch of the threads and a desired insertion depth, to ensure that the slot of the anchor aligns with the slot formed in the phalange when the anchor is screwed in to the desired insertion depth.
75. A surgical procedure for replacing a flexible connector of an implanted device for arthroplasty, comprising:
   identifying a patient having an already-implanted device for arthroplasty comprising two anchors as in any of examples 48-66 and a flexible connector as in any of examples 1-42 held between the two anchors;
performing an incision at the implant site of the patient;
removing the flexible connector from the anchors;
fitting a replacement flexible connector, wherein the replacement flexible connector comprises anchoring portions compatible with the anchors; and
performing a surgical closure of a wound at the implant site.
76. An implantable device for arthroplasty, comprising:
   a first anchor and a second anchor, wherein each anchor comprises:
   a hollow anchor body defining an interior canula space,
   a slot extending along a length of the anchor body, the slot providing an opening from the interior cannula space to an exterior of the anchor body,
wherein the slot has a slot width that is smaller than an internal width dimension in the interior cannula space, and
   a rounded edge at a transition between the slot and an interior surface of the anchor body, or between the slot and an exterior surface of the anchor body, or both; and
a flexible connector comprising:
a flexible central portion configured to extend through the slots of the anchors,
   a first anchoring portion extending from a first end of the central portion and configured to be held in the interior cannula space of the first anchor, and
a second anchoring portion extending from a second end of the central portion, opposite from the first end, and configured to be held in the interior cannula space of the second anchor, wherein the first anchoring portion and the second anchoring portion each comprise a thickened portion located farther away from the central portion and a thinner portion, relative to the thickened portion, located nearer to the central portion than the thickened portion.
77. The device of example 76, wherein the interior cannula space and rounded edges of the first anchor and the second anchor form a teardrop-shaped profile, and wherein the first anchoring portion and the second anchoring portion have a teardrop-shaped profile to conform to the first anchor and the second anchor.
78. The device of any of examples 76-77, wherein the interior cannula space of the first anchor and the second anchor comprises a non-uniform internal diameter profile along a length of the anchor, and wherein the first anchoring portion and the second anchoring portion have a non-uniform thickness profile shaped to conform to the non-uniform internal diameter profile to facilitate retention of the first anchoring portion in the first anchor and the second anchoring portion in the second anchor.
79. The device of any of examples 76-78, wherein the first anchor and the second anchor are devoid of sharp edges in locations that contact the flexible connector during flexion and extension of a joint into which the device is implanted.
80. The device of any of examples 77-79, wherein the flexible connector has a first width adjacent to at least one of the first anchoring portion or the second anchoring portion to fit within at least one of the first anchor or the second anchor, and wherein the width of the flexible connector increases from the first width to a greater second width farther from the first anchoring portion or the second anchoring portion.
81. The device of example 80, wherein the second width is substantially a full width of a joint capsule into which the device is to be implanted.
82. The device of example 76, wherein at least one of the first anchor or the second anchor is an anchor according to any of examples 49-67 and wherein the flexible connector is a flexible connector according to any of examples 1-42.

While the foregoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention.

The term “comprising” is used throughout to mean including at least the recited feature(s) such that any greater number of the same feature and/or additional types of other features are not precluded. It will also be appreciated that specific embodiments have been described herein for purposes of illustration, but that various modifications can be made without deviating from the technology. Further, while advantages associated with some embodiments of the present technology have been described in the context of those embodiments, other embodiments can also exhibit such advantages, and not all embodiments need necessarily exhibit such advantages to fall within the scope of the technology. Accordingly, the disclosure and associated present technology can encompass other embodiments not expressly shown or described herein.

Moreover, unless the word “or” is expressly limited to mean only a single item exclusive from other items in reference to a list of two or more items, then the use of “or” in such a list is to be interpreted as including (a) any single item in the list, (b) all of the items in the list, or (c) any combination of the items in the list. In other words, the use of “or” in this disclosure should be understood to mean non-exclusive “or” (i.e., “and/or”) unless otherwise indicated herein.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described present technology

What is claimed is:

1. An anchor for an implantable device for arthroplasty, comprising:
a hollow anchor body defining an interior cannula space;
a slot extending along a length of the anchor body, the slot providing an opening from the interior cannula space to an exterior of the anchor body, wherein the slot has a slot width that is smaller than an internal width dimension in the interior cannula space; and
a rounded edge at a transition between the slot and at least one of an interior surface of the anchor body or an exterior surface of the anchor body.

2. The anchor of claim 1, wherein the rounded edge is only present along one side of the slot.

3. The anchor of claim 1, wherein the rounded edge is present along both sides of the slot.

4. The anchor of claim 1, wherein the rounded edge comprises rounded edges present at the transitions from the slot to the interior surface along both sides of the slot, and wherein the rounded edges and the interior cannula space together have a teardrop-shaped profile.

5. The anchor of claim 1, wherein the interior cannula space comprises a non-uniform internal diameter profile along a length of the anchor to facilitate retention of a flexible connector having an anchoring portion shaped to conform to the non-uniform internal diameter profile.

6. The anchor of claim 5, wherein the non-uniform internal diameter profile comprises a narrowed portion at one end or both ends of the anchor and a larger-diameter portion between the ends.

7. The anchor of claim 1, wherein the exterior surface of the anchor body has a smooth, cylindrical shape.

8. The anchor of claim 1, wherein the exterior surface of the anchor body has a cross-sectional profile selected from circular, square, triangular, hexagonal, oval, or a dovetail groove shape.

9. The anchor of claim 1, wherein the exterior surface of the anchor body comprises threads configured to allow the anchor body to be screwed into a phalangeal bone.

10. The anchor of claim 1, wherein the threads are discontinuous at the slot.

11. The anchor of claim 10, wherein a depth of the threads decreases gradually toward the slot such that the threads do not protrude from the exterior surface of the anchor body adjacent to the slot.

12. The anchor of claim 10, wherein the threads comprise at least one other discontinuity at another location besides the slot.

13. The anchor of claim 1, wherein the anchor body is configured to be implanted transversely through an entire width of a phalangeal bone.

14. The anchor of claim 1, further comprising a stem extending from the exterior surface of the anchor body, opposite from the slot and orthogonal to the interior cannula space.

15. The anchor of claim 1, further comprising a screw hole in the anchor body, the screw hole extending from the interior surface to the exterior surface, the screw hole being positioned opposite from the slot.

16. The anchor of claim 15, further comprising a fixation screw configured to be screwed through the screw hole into a phalangeal bone along the length of the phalangeal bone.

17. The anchor of claim 16, wherein the fixation screw increases in diameter toward the anchor when the screw is screwed in place.

18. The anchor of claim 16, wherein the fixation screw comprises a hole extending transversely through a shaft of the screw to allow a pin to be inserted through the hole after the screw has been screwed in place.

19. The anchor of claim 16, wherein the fixation screw comprises two holes extending through a shaft of the screw, wherein the two holes are orthogonal one to another, to allow a pin to be inserted transversely through one of the holes after the screw has been screwed in place.

20. A surgical procedure for implanting an implantable device for arthroplasty, comprising:
selecting a patient in need of implantation of an implantable device for arthroplasty;
providing two anchors as in claim 1;
providing a flexible connector configured to be held between the two anchors; and
implanting the anchors and the flexible connector into the patient.

21. The surgical procedure of claim 20, further comprising:
performing an incision at an implant site of the patient;
preparing a joint capsule and two phalangeal bones at the implant site;
fitting the anchors into the two phalangeal bones;
fitting the flexible connector into the anchors; and
performing a surgical closure of a wound at the implant site.

22. The surgical procedure of claim 21, wherein preparing the two phalangeal bones comprises:
forming a first cavity in a proximal phalange optionally using a surgical guide;
forming a second cavity in a distal phalange optionally using a surgical guide;
forming a first slot in the proximal phalange and a second slot in the distal phalange, optionally using a cutting guide, wherein the first slot and the second slot face one another;
wherein a proximal anchor of the two anchors is fit transversely into the first cavity and a distal anchor of the two anchors is fit transversely into the second cavity such that slots of the anchors are aligned with the first slot and the second slot.

23. The surgical procedure of claim 22, wherein the flexible connector comprises a first anchoring portion held in the proximal anchor, a second anchoring portion held in the distal anchor, and a flexible central portion extending from the first anchoring portion, through the slots of the anchors and the proximal and second slots of the phalangeal bones, to the second anchoring portion.

24. The surgical procedure of claim 22, wherein the proximal anchor is press-fit into the first cavity, or wherein the distal anchor is press-fit into the second cavity, or both.

25. The surgical procedure of claim 22, wherein at least one of the proximal anchor or the distal anchor comprises threads on an exterior surface, and wherein at least one of the proximal anchor or the distal anchor is fit by screwing into bone.

26. The surgical procedure of claim 25, further comprising pre-calculating a starting rotational position for at least one of the proximal anchor or the distal anchor based on a pitch of the threads and a desired insertion depth, to ensure that the slot of the anchor aligns with the slot formed in the phalange when the anchor is screwed in to the desired insertion depth.

27. A surgical procedure for replacing a flexible connector of an implanted device for arthroplasty, comprising:
identifying a patient having an already-implanted device for arthroplasty comprising two anchors as in claim 1 and a flexible connector held between the two anchors;
performing an incision at the implant site of the patient;
removing the flexible connector from the anchors;
fitting a replacement flexible connector, wherein the replacement flexible connector comprises anchoring portions compatible with the anchors; and
performing a surgical closure of a wound at the implant site.

28. An implantable device for arthroplasty, comprising:
a first anchor and a second anchor, wherein each anchor comprises:
a hollow anchor body defining an interior canula space,
a slot extending along a length of the anchor body, the slot providing an opening from the interior cannula space to an exterior of the anchor body, wherein the slot has a slot width that is smaller than an internal width dimension in the interior cannula space, and
a rounded edge at a transition between the slot and an interior surface of the anchor body, or between the slot and an exterior surface of the anchor body, or both; and
a flexible connector comprising:
a flexible central portion configured to extend through the slots of the anchors,
a first anchoring portion extending from a first end of the central portion and configured to be held in the interior cannula space of the first anchor, and
a second anchoring portion extending from a second end of the central portion, opposite from the first end, and configured to be held in the interior cannula space of the second anchor, wherein the first anchoring portion and the second anchoring portion each comprise a thickened portion located farther away from the central portion and a thinner portion, relative to the thickened portion, located nearer to the central portion than the thickened portion.

29. The device of claim 28, wherein the interior cannula space and rounded edges of the first anchor and the second anchor form a teardrop-shaped profile, and wherein the first anchoring portion and the second anchoring portion have a teardrop-shaped profile to conform to the first anchor and the second anchor.

30. The device of claim 28, wherein the interior cannula space of the first anchor and the second anchor comprises a non-uniform internal diameter profile along a length of the anchor, and wherein the first anchoring portion and the second anchoring portion have a non-uniform thickness profile shaped to conform to the non-uniform internal diameter profile to facilitate retention of the first anchoring portion in the first anchor and the second anchoring portion in the second anchor.

31. The device of claim 28, wherein the first anchor and the second anchor are devoid of sharp edges in locations that contact the flexible connector during flexion and extension of a joint into which the device is implanted.

32. The device of claim 28, wherein the flexible connector has a first width adjacent to at least one of the first anchoring portion or the second anchoring portion to fit within at least one of the first anchor or the second anchor, and wherein the width of the flexible connector increases from the first width to a greater second width farther from the first anchoring portion or the second anchoring portion.

33. The device of claim 32, wherein the second width is substantially a full width of a joint capsule into which the device is to be implanted.
